(19) United States
(12) Patent Application Publication
(10) Pub. No.: US 2022/0388969 A1
(43) Pub. Date: Dec. 8, 2022

(54) SUBSTITUTED 3,4-DIHYDROQUINAZOLINE FOR THE TREATMENT AND PROPHYLAXIS OF HEPATITIS B VIRUS INFECTION

(71) Applicant: Hoffmann-La Roche Inc., Little Falls, NJ (US)

(72) Inventors: Dongdong CHEN, Shanghai (CN); Wenming CHEN, Shanghai (CN); Song FENG, Shanghai (CN); Chao LI, Shanghai (CN); Zongxing QIU, Shanghai (CN); Xuefei TAN, Shanghai (CN); Guolong WU, Shanghai (CN)

(73) Assignee: Hoffmann-La Roche Inc., Little Falls, NJ (US)

(21) Appl. No.: 17/765,378

(22) PCT Filed: Sep. 28, 2020
(86) PCT No.: PCT/EP2020/077041

§ 371 Date: Mar. 30, 2022

(30) Foreign Application Priority Data
Sep. 30, 2019 (CN) PCT/CN2019/109510

(51) Int. Cl. C07D 239/74

(52) U.S. Cl. CPC C07D 239/74

(57) ABSTRACT

The present invention provides novel compounds having the general formula (I) wherein R¹ to R⁶ are as described herein, compositions including the compounds and methods of using the compounds.

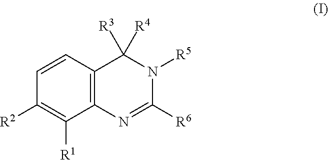

The present invention relates to organic compounds useful for therapy and/or prophylaxis of HBV infection in a mammal, and in particular to cccDNA (covalently closed circular DNA) inhibitors useful for treating HBV infection.

### FIELD OF THE INVENTION

The present invention relates to substituted 3,4-dihydroquinazoline having pharmaceutical activity, their manufacture, pharmaceutical compositions containing them and their potential use as medicaments.

The present invention relates to compounds of formula (I)

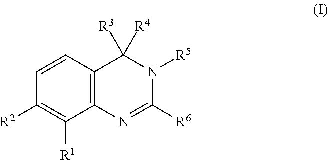

wherein R¹ to R⁶ are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R.G. et al., Antiviral Res (2015), 121, 47-58; Levrero, M. et al., J Hepatol (2009), 51 (3), 581-592.). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are compounds of formula (I), their manufacture, medicaments based on a compound in accordance with the invention and their production as well as the use of compounds of formula (I) as cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

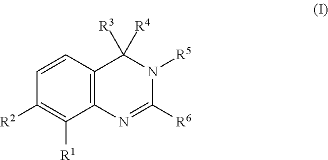

wherein
R¹ is H, halogen, C₁₋₆alkyl or haloC₁₋₆alkyl;
R² is H, halogen, C₁₋₆alkyl or haloC₁₋₆alkyl;
R³ is H, carboxy, C₁₋₆alkyl, haloC₁₋₆alkyl, hydroxyC₁₋₆alkyl, C₁₋₆alkoxycarbonyl, C₃₋₇cycloalkyl, aminocarbonyl, hydroxyC₁₋₆alkylaminocarbonyl, haloC₁₋₆alkylaminocarbonyl or heterocyclylcarbonyl;
R⁴ is H or C₁₋₆alkyl;
R⁵ is H, C₁₋₆alkyl or hydroxyC₁₋₆alkyl;
R⁶ is phenyl or heterocyclyl; wherein phenyl and heterocyclyl are unsubstituted or substituted by one or two or three substituents independently selected from halogen, C₁₋₆alkyl, C₁₋₆alkoxy, hydroxyC₁₋₆alkoxy, C₁₋₆alkoxycarbonylphenyl, carboxyC₁₋₆alkoxyC₁₋₆alkoxy, carboxyC₃₋₇cycloalkylC₁₋₆alkoxy and heterocyclyl;
with the proviso that R¹ and R² are not H simultaneously;
or a pharmaceutically acceptable salt thereof.

### DETAILED DESCRIPTION OF THE INVENTION

### DEFINITIONS

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl. Most particular “C₁₋₆alkyl” group is methyl.

The term “C₁₋₆alkoxy” alone or in combination signifies a group C₁₋₆alkyl—O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” groups are cyclopropyl and cyclobutyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro or chloro atoms. Examples of haloC₁₋₆alkyl include monochloro-, difluoro-or trifluoro-methyl, -ethyl or -propyl, for example difluoromethyl, chloroethyl and trifluoromethyl.

“Heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Examplary heterocyclyls are pyridyl, 1-oxo-3,4-dihydroisoquinolin-2-yl, imidazolyl, morpholino, pyrrolidinyl and thiazolyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, hydroxyC₁₋₆alkoxy, C₁₋₆alkoxycarbonylphenyl, carboxyC₁₋₆alkoxyC₁₋₆alkoxy, carboxyC₃₋₇cycloalkylC₁₋₆alkoxy, phenyl or heterocyclyl.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R.J., et al., Organic Process Research & Development 2000, 4, 427-435. Particular are the sodium salts of the compounds of formula (I).

### HBV INHIBITORS

The present invention provides (i) a compound having the general formula (I):

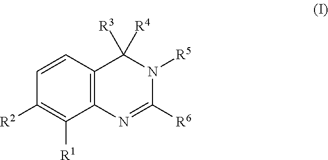

wherein
R¹ is H, halogen, C₁₋₆alkyl or haloC₁₋₆alkyl;
R² is H, halogen, C₁₋₆alkyl or haloC₁₋₆alkyl;
R³ is H, carboxy, C₁₋₆alkyl, haloC₁₋₆alkyl, hydroxyC₁₋₆alkyl, C₁₋₆alkoxycarbonyl, C₃₋₇cycloalkyl, aminocarbonyl, hydroxyC₁₋₆alkylaminocarbonyl, haloC₁₋₆alkylaminocarbonyl or heterocyclylcarbonyl;
R⁴ is H or C₁₋₆alkyl;
R⁵ is H, C₁₋₆alkyl or hydroxyC₁₋₆alkyl;
R⁶ is phenyl or heterocyclyl; wherein phenyl and heterocyclyl are unsubstituted or substituted by one or two or three substituents independently selected from halogen, C₁₋₆alkyl, C₁₋₆alkoxy, hydroxyC₁₋₆alkoxy, C₁₋₆alkoxycarbonylphenyl, carboxyC₁₋₆alkoxyC₁₋₆alkoxy, carboxyC₃₋₇cycloalkylC₁₋₆alkoxy and heterocyclyl;
with the proviso that R¹ and R² are not H simultaneously;
or a pharmaceutically acceptable salt thereof.

A further embodiment of the present invention is (ii) a compound of formula (I) according to (i), wherein
R¹ is H or halogen;
R² is H or haloC₁₋₆alkyl;
R³ is H, carboxy, C₁₋₆alkyl, hydroxyC₁₋₆alkyl, C₁₋₆alkoxycarbonyl, C₃₋₇cycloalkyl, aminocarbonyl, hydroxyC₁₋₆alkylaminocarbonyl, haloC₁₋₆alkylaminocarbonyl or morpholinocarbonyl;
R⁴ is H or C₁₋₆alkyl;
R⁵ is H, C₁₋₆alkyl or hydroxyC₁₋₆alkyl;
R⁶ is phenyl, pyridyl, 1-oxo-3,4-dihydroisoquinolin-2-yl, imidazolyl or thiazolyl; wherein phenyl, pyridyl, 1-oxo-3,4-dihydroisoquinolin-2-yl, imidazolyl and thiazolyl are unsubstituted or substituted by one or two or three substituents independently selected from halogen, C₁₋₆alkyl, C₁₋₆alkoxy, pyrrolidinyl, hydroxyC₁₋₆alkoxy, C₁₋₆alkoxycarbonylphenyl, carboxyC₁₋₆alkoxyC₁₋₆alkoxy and carboxyC₃₋₇cycloalkylC₁₋₆alkoxy;
with the proviso that R¹ and R² are not H simultaneously;
or a pharmaceutically acceptable salt thereof.

A further embodiment of the present invention is (iii) a compound of formula (I) according to any one of (i)-(ii), wherein
R¹ is H or Cl;
R² is H or CF₃;
R³ is H, carboxy, methyl, isopropyl, hydroxymethyl, methoxycarbonyl, cyclopropyl, aminocarbonyl, hydroxyethylaminocarbonyl, chloroethylaminocarbonyl or morpholinocarbonyl;
R⁴ is H or methyl;
R⁵ is H, methyl or hydroxyethyl;
R⁶ is phenyl, pyridyl, 1-oxo-3,4-dihydroisoquinolin-2-yl, imidazolyl or thiazolyl; wherein phenyl, pyridyl, 1-oxo-3,4-dihydroisoquinolin-2-yl, imidazolyl and thiazolyl are unsubstituted or substituted by one or two or three substituents independently selected from Cl, Br, methyl, methoxy, ethoxy, pyrrolidinyl, hydroxyethoxy, methoxycarbonylphenyl, carboxymethoxyethoxy and carboxycyclobutoxyethoxy;
with the proviso that R¹ and R² are not H simultaneously;
or a pharmaceutically acceptable salt thereof.

A further embodiment of the present invention is (iv) a compound of formula (I) according to any one of (i)-(iii), or a pharmaceutically acceptable salt thereof, wherein R³ is carboxy, C₁₋₆alkyl or aminocarbonyl.

A further embodiment of the present invention is (v) a compound of formula (I) according to any one of (i)-(iv), or a pharmaceutically acceptable salt thereof, wherein R³ is carboxy, methyl, isopropyl or aminocarbonyl.

A further embodiment of the present invention is (vi) a compound of formula (I) according to any one of (i)-(v), or a pharmaceutically acceptable salt thereof, wherein R⁶ is phenyl, imidazolyl or thiazolyl; wherein phenyl, imidazolyl and thiazolyl are unsubstituted or substituted by one or two substituents independently selected from halogen, C₁₋₆alkyl, C₁₋₆alkoxy, C₁₋₆alkoxycarbonylphenyl and carboxyC₃₋₇cycloalkylC₁₋₆alkoxy.

A further embodiment of the present invention is (vii) a compound of formula (I) according to any one of (i)-(vi), or a pharmaceutically acceptable salt thereof, wherein R⁶ is phenyl, imidazolyl or thiazolyl; wherein phenyl, imidazolyl and thiazolyl are unsubstituted or substituted by one or two substituents independently selected from Br, methyl, methoxy, methoxycarbonylphenyl and carboxycyclobutoxyethoxy.

A further embodiment of the present invention is (viii) a compound of formula (I) according to any one of (i)-(vii), or a pharmaceutically acceptable salt thereof, wherein R⁴ is H.

A further embodiment of the present invention is (ix) a compound of formula (I) according to any one of (i)-(viii), or a pharmaceutically acceptable salt thereof, wherein R⁵ is H.

A further embodiment of the present invention is (x) a compound of formula (II) according to any one of (i)-(ix),

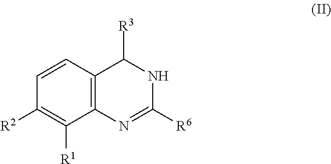

wherein
R¹ is H or halogen;
R² is H or haloC₁₋₆alkyl;
R³ is carboxy, C₁₋₆alkyl or aminocarbonyl;
R⁶ is phenyl, imidazolyl or thiazolyl; wherein phenyl, imidazolyl and thiazolyl are unsubstituted or substituted by one or two substituents independently selected from halogen, C₁₋₆alkyl, C₁₋₆alkoxy, C₁₋₆alkoxycarbonylphenyl and carboxyC₃₋₇cycloalkylC₁₋₆alkoxy;
with the proviso that R¹ and R² are not H simultaneously;
or a pharmaceutically acceptable salt thereof.

A further embodiment of the present invention is (xi) a compound of formula (II) according to any one of (i)-(x), wherein
R¹ is H or Cl;
R² is H or CF_{3;}
R³ is carboxy, methyl, isopropyl or aminocarbonyl;
R⁶ is phenyl, imidazolyl or thiazolyl; wherein phenyl, imidazolyl and thiazolyl are unsubstituted or substituted by one or two substituents independently selected from Br, methyl, methoxy, methoxycarbonylphenyl and carboxycyclobutoxyethoxy;
with the proviso that R¹ and R² are not H simultaneously;
or a pharmaceutically acceptable salt thereof.

In another embodiment (xii) of the present invention, particular compounds of the present invention are selected from:

2-(4-bromophenyl)-8-chloro-3,4-dihydroquinazoline-4-carboxylic acid;
8-chloro-2-phenyl-3,4-dihydroquinazoline-4-carboxylic acid;
8-chloro-2-(4-chlorophenyl)-3,4-dihydroquinazoline-4-carboxylic acid;
8-chloro-2-(4-methoxyphenyl)-3,4-dihydroquinazoline-4-carboxylic acid;
2-(4-bromo-3-methyl-phenyl)-8-chloro-3,4-dihydroquinazoline-4-carboxylic acid;
2-(3-bromophenyl)-7-(trifluoromethyl)-3,4-dihydroquinazoline-4-carboxylic acid;
2-(4-bromophenyl)-7-(trifluoromethyl)-3,4-dihydroquinazoline-4-carboxylic acid;
8-chloro-2-(6-methoxy-3-pyridyl)-3,4-dihydroquinazoline-4-carboxylic acid;
8-chloro-2-(6-ethoxy-3-pyridyl)-3,4-dihydroquinazoline-4-carboxylic acid;
8-chloro-2-(6-pyrrolidin-l-yl-3-pyridyl)-3,4-dihydroquinazoline-4-carboxylic acid;
2-(4-bromophenyl)-8-chloro-4-methyl-3H-quinazoline-4-carboxylic acid;
methyl 2-(4-bromophenyl)-8-chloro-3,4-dihydroquinazoline-4-carboxylate;
2-(4-bromophenyl)-8-chloro-3,4-dihydroquinazoline-4-carboxamide;
8-chloro-2-(4-methoxyphenyl)-3,4-dihydroquinazoline-4-carboxamide;
2-(3-bromophenyl)-7-(trifluoromethyl)-3,4-dihydroquinazoline-4-carboxamide;
8-chloro-N-(2-hydroxyethyl)-2-(4-methoxyphenyl)-3,4-dihydroquinazoline-4-carboxamide;
[2-(4-bromophenyl)-7-(trifluoromethyl)-3,4-dihydroquinazolin-4-yl]-morpholino-methanone;
2-(4-bromophenyl)-8-chloro-N-(2-chloroethyl)-3,4-dihydroquinazoline-4-carboxamide;
3-[2-[4-[4-methyl-7-(trifluoromethyl)-3,4-dihydroquinazolin-2-yl]phenoxy]ethoxy]cyclobutanecarboxylic acid;
2-[2-[4-[4-methyl-7-(trifluoromethyl)-3,4-dihydroquinazolin-2-yl]phenoxy]ethoxy]acetic acid;
3-[2-[4-(8-chloro-4-methyl-3,4-dihydroquinazolin-2-yl)phenoxy]ethoxy]cyclobutanecarboxylic acid;
3-[2-[4-(8-chloro-4-methyl-3,4-dihydroquinazolin-2-yl)-3-methyl-phenoxy]ethoxy]cyclobutanecarboxylic acid;
2-[2-[4-(8-chloro-4-methyl-3,4-dihydroquinazolin-2-yl)phenoxy]ethoxy]acetic acid;
2-(3-bromophenyl)-3,4-dimethyl-7-(trifluoromethyl)-4H-quinazoline;
2-[2-(3-bromophenyl)-4-methyl-7-(trifluoromethyl)-4H-quinazolin-3-yl]ethanol;
2-[2-[4-(8-chloro-3,4-dimethyl-4H-quinazolin-2-yl)phenoxy]ethoxy]acetic acid;
2-[4-(8-chloro-3,4-dihydroquinazolin-2-yl)phenoxy]ethanol;
2-[4-(8-chloro-4-methyl-3,4-dihydroquinazolin-2-yl)phenoxy]ethanol;
2-(8-chloro-4-methyl-3,4-dihydroquinazolin-2-yl)-6-methoxy-3,4-dihydroisoquinolin-l-one;
2-(4-bromoimidazol-1-yl)-4-methyl-7-(trifluoromethyl)-3,4-dihydroquinazoline;
4-bromo-2-(8-chloro-4-methyl-3,4-dihydroquinazolin-2-yl)thiazole;
methyl 4-[2-(8-chloro-4-methyl-3,4-dihydroquinazolin-2-yl)thiazol-4-yl]benzoate;
2-(3-bromophenyl)-4-methyl-7-(trifluoromethyl)-3,4-dihydroquinazoline;
2-(4-bromophenyl)-8-chloro-4,4-dimethyl-3H-quinazoline;
2-(4-bromophenyl)-8-chloro-4-cyclopropyl-3,4-dihydroquinazoline;
2-(4-bromophenyl)-8-chloro-4-isopropyl-3,4-dihydroquinazoline;
3-[2-[5-bromo-2-(8-chloro-4-methyl-3,4-dihydroquinazolin-2-yl)phenoxy]ethoxy]cyclobutanecarboxylic acid; and
[2-(4-bromophenyl)-8-chloro-3,4-dihydroquinazolin-4-yl]methanol; or a pharmaceutically acceptable salt thereof.

In another embodiment (xiii) of the present invention, particular compounds of the present invention are selected from:

8-chloro-2-phenyl-3,4-dihydroquinazoline-4-carboxylic acid;
8-chloro-2-(4-methoxyphenyl)-3,4-dihydroquinazoline-4-carboxylic acid;
2-(4-bromophenyl)-8-chloro-3,4-dihydroquinazoline-4-carboxamide;
3-[2-[4-(8-chloro-4-methyl-3,4-dihydroquinazolin-2-yl)-3-methyl-phenoxy]ethoxy]cyclobutanecarboxylic acid;
2-(4-bromoimidazol-1-yl)-4-methyl-7-(trifluoromethyl)-3,4-dihydroquinazoline; methyl 4-[2-(8-chloro-4-methyl-3,4-dihydroquinazolin-2-yl)thiazol-4-yl]benzoate; and
2-(4-bromophenyl)-8-chloro-4-isopropyl-3,4-dihydroquinazoline;
or a pharmaceutically acceptable salt thereof.

### SYNTHESIS

The compounds of the present invention can be prepared by any conventional means. Suitable processes for synthesizing these compounds as well as their starting materials are provided in the schemes below and in the examples. All substituents, in particular, R¹ to R⁶ are as defined above unless otherwise indicated. Furthermore, and unless explicitly otherwise stated, all reactions, reaction conditions, abbreviations and symbols have the meanings well known to a person of ordinary skill in organic chemistry.

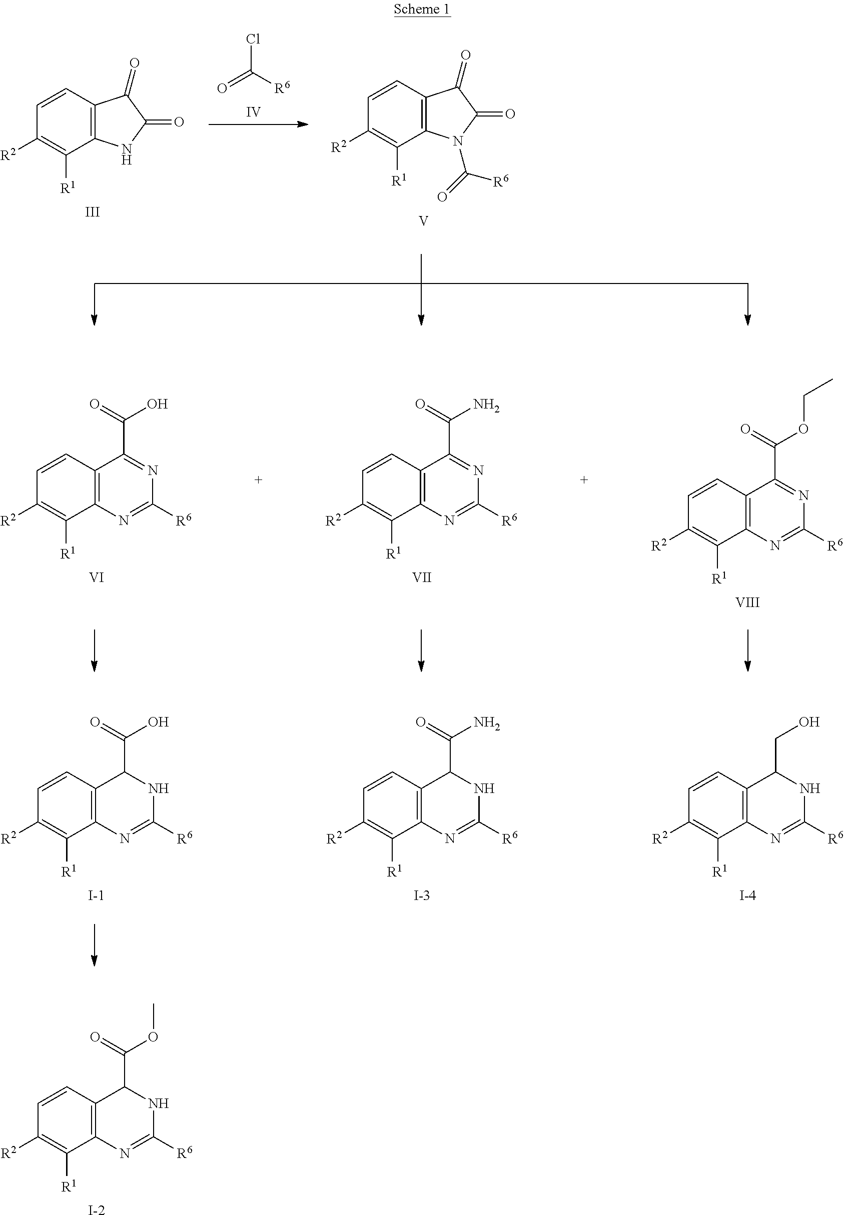

Acylation of a compound of formula III with a compound of formula IV in a suitable solvent, such as pyridine, affords a compound of formula V. Treatment of a compound of formula V with an ammonium acetate in a suitable solvent, such as EtOH, affords a mixture of acid of formula VI, amide of formula VII and ester of formula VIII. Reduction of a compound of formula VI with a suitable reductant, such as NaBH₄, in the presence of a suitable solvent, such as MeOH, affords a compound of formula I-1. Esterification of a compound of formula I-1 with a suitable reagent, such as MeI, in the presence of a suitable base, such as K₂CO₃, in a suitable solvent, such as DMF, affords compound of formula I-2. Reduction of a compound of formula VII with a suitable reductant, such as NaBH₄, in the presence of a suitable solvent, such as MeOH, affords a compound of formula I-3. Reduction of a compound of formula VIII with a suitable reductant, such as LiAlH₄, in the presence of a suitable solvent, such as THF, affords a compound of formula I-4.

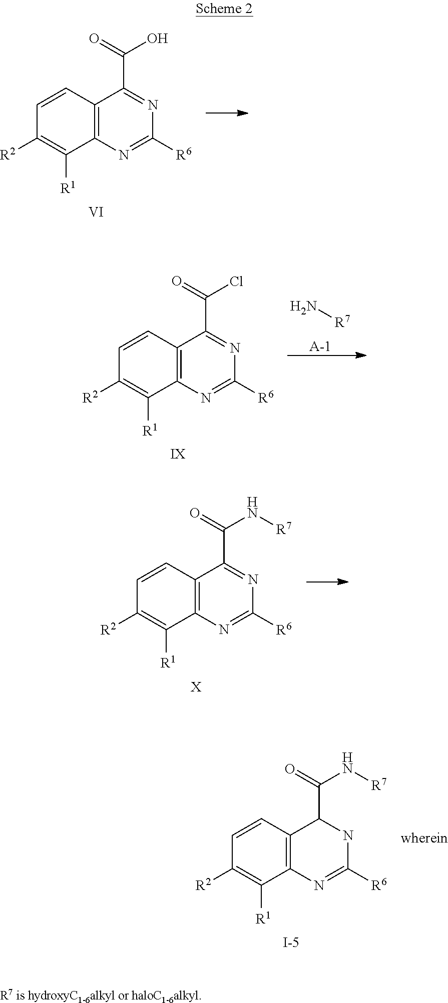

Treatment of a compound of formula VI with oxalyl dichloride in a suitable solvent, such as DCM, affords a compound of formula IX. Treatment of a compound of formula IX with a suitable amine A-1, such as 2-aminoethanol, in the presence of a suitable base, such as TEA, in a suitable solvent, such as DCM, affords a compound of formula X. Reduction of a compound of formula X with a suitable reductant, such as NaBH₄, in the presence of a suitable solvent, such as THF, affords a compound of formula I-5.

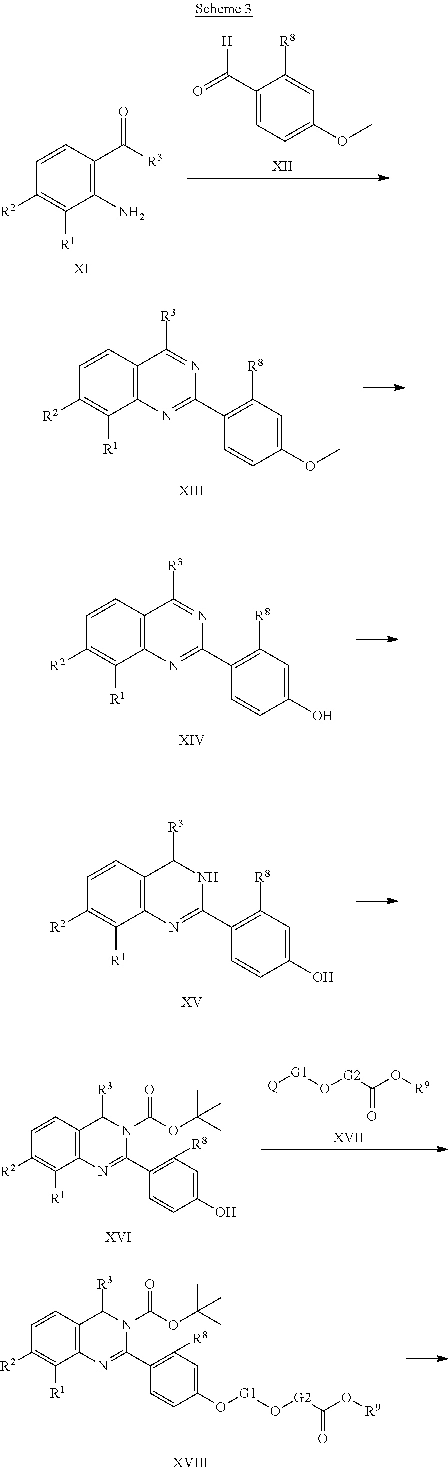

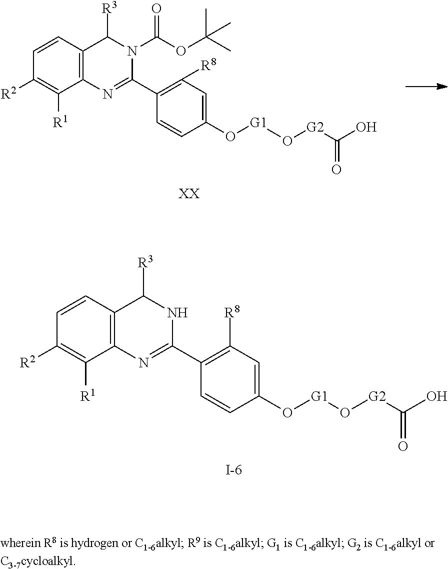

Cyclization of a compound of formula XI with aldehyde XII with a suitable oxidant, such as tert-Butyl hydroperoxide, ammonium cerium(IV) nitrate in an appropriate solvent, such as acetonitrile, affords a compound of formula XIII. Demethylation of a compound of formula XIII with a suitable Lewis acid, such as boron tribromide, in a suitable solvent, such as DCM, affords a compound of formula XIV. Reduction of a compound of formula XIV with a suitable reductant, such as NaBH₄, in the presence of a suitable solvent, such as MeOH, affords a compound of formula XV. Protection an amino group in a compound of formula XV with di-t-butyldicarbonate in the presence of a suitable base, such as TEA, in a suitable solvent, such as DCM, affords a compound of formula XVI. Substitution of a compound of formula XVI with a compound of formula XVII in the presence of a suitable base, such as K₂CO₃, in a suitable solvent, such as ACN, affords a compound of formula XVIII. Hydrolysis of a compound of formula XVIII with a suitable base, such as trimethylstannanol, in a suitable solvent, such as DCE, affords a compound of formula XX. Deprotection of a compound of formula XX with a suitable acid, such as TFA, in a suitable solvent, such as DCM, affords a compound of formula I-6.

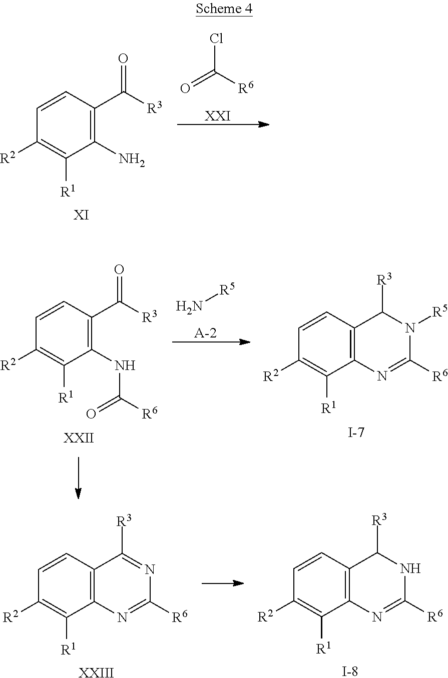

Acylation of a compound XI with acyl chloride of formula XXI in a suitable solvent, such as pyridine, affords a compound of formula XXII. Treatment of a compound of formula XXII with a suitable amine of formula A-2, such as methanamine, in a suitable solvent, such as EtOH, then followed with a suitable reductant, such as NaBH₄, affords a compound of formula I-7. Treatment of a compound of formula XXII with ammonium acetate in a suitable solvent, such as EtOH, affords a compound of formula XXIII. Reduction of a compound of formula XXIII with a suitable reductant, such as NaBH₄, in the presence of a suitable solvent, such as MeOH, affords a compound of formula I-8.

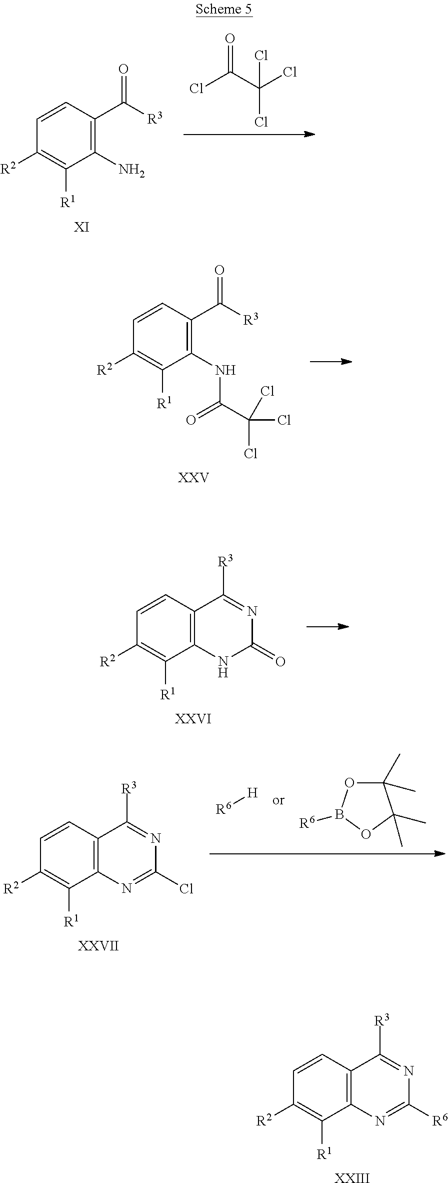

Compound XXIII can also be obtained via Scheme 5. Treatment of a compound of formula XI with 2,2,2-trichloroacetyl chloride in the presence of a suitable base, such as DMAP, in an appropriate solvent, such as DCM, affords a compound of formula XXV. Cyclization of a compound of formula XXV with ammonium hydroxide in a suitable solvent, such as THF, affords a compound of formula XXVI. Treatment of a compound of formula XXVI with POCl₃ in the presence of a suitable base, such as N,N-diethylaniline affords a compound of formula XXVII. Substitution of a compound of formula XXVII with a heterocycle, such as 4-bromo-1H-imidazole, in the presence of a suitable base, such as K₂CO₃, in an appropriate solvent, such as DMF, affords a compound of formula XXIII. The compound of formula XXIII can also be obtained by coupling a compound of formula XXVII with a borate esters, such as 2-(4-(4,4,5,5-tetramethyl-1,3,2-dioxaborolan-2-yl)phenoxy)ethanol, in the presence of a suitable catalyst, such as Pd(Ph₃P)₄ and a suitable base, such as Cs₂CO₃, in an appropriate solvent, such as dioxane.

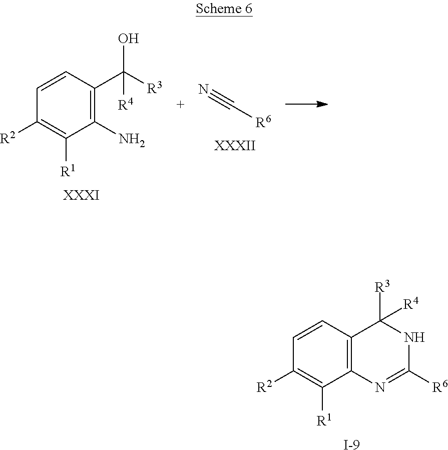

Cyclization of a compound of formula XXXI with a compound of formula XXXII with a suitable Lewis acid, such as BF₃.Et₂O, affords a compound of formula I-9.

This invention also relates to a process for the preparation of a compound of formula (I) comprising at least one of the following steps:

(a) reduction of a compound of formula (VI),

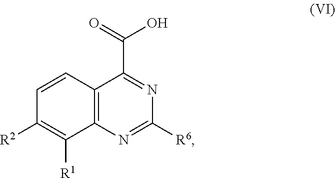

with a reductant;
(b) esterification of a compound of formula (I-1),

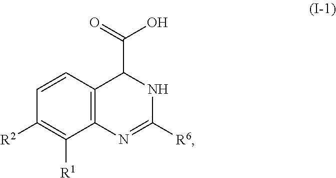

with MeI, in the presence of a base;
(c) reduction of a compound of formula (VII),

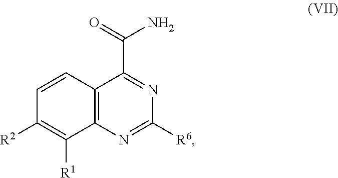

with a reductant;
(d) reduction of a compound of formula (VIII),

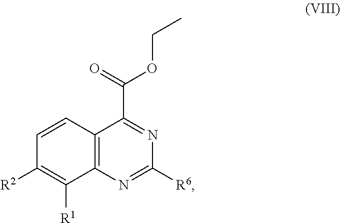

with a reductant;
(e) reduction of a compound of formula (X),

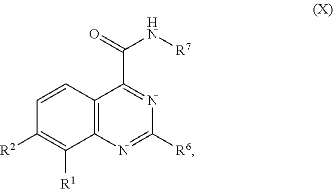

with a reductant;
(f) deprotection of a compound of formula (XX),

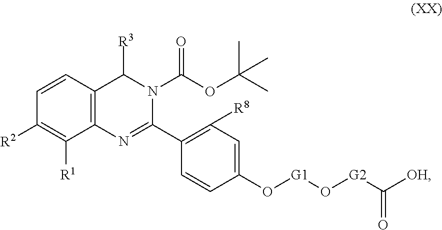

with an acid;
(g) treatment of a compound of formula (XXII),

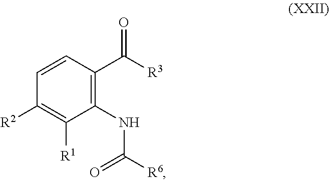

with an amine of formula (A-2),

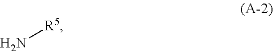

in the presence of a reductant;
(h) reduction of a compound of formula (XXIX),

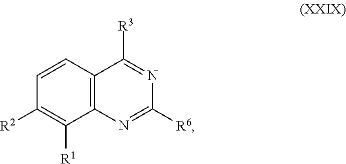

with a reductant;
(i) cyclization of a compound of formula (XXXI),

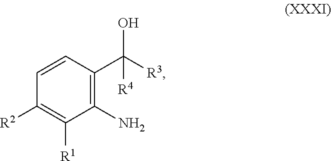

with a compound of formula (XXXII),

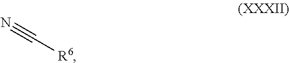

in the presence a Lewis acid;
wherein R⁷ is hydroxyC₁₋₆alkyl or haloC₁₋₆alkyl; R⁸ is hydrogen or C₁₋₆alkyl; R⁹ is C₁₋₆alkyl; G₁ is C₁₋₆alkyl; G₂ is C₁₋₆alkyl or C₃₋₇cycloalkyl.
The reductant in step (a), (c), (e), (g) or (h) can be, for example, NaBH₄.
The base in step (b) can be, for example, K₂CO₃.
The reductant in step (d) can be, for example, LiAlH₄.
The acid in step (f) can be, for example, TFA.
The Lewis acid in step (i) can be, for example, BF₃.Et₂O.

A compound of formula (I) or (II) when manufactured according to the above process is also an object of the invention.

### PHARMACEUTICAL COMPOSITIONS AND ADMINISTRATION

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I) or (II), or pharmaceutically acceptable salt thereof.

In a further embodiment includes a pharmaceutical composition comprising a compound of formula (I) or (II), or pharmaceutically acceptable salt thereof, together with a pharmaceutically acceptable carrier or excipient.

Another embodiment includes a pharmaceutical composition comprising a compound of formula (I) or (II), or pharmaceutically acceptable salt thereof, for use in the treatment of HBV infection.

### INDICATIONS AND METHODS OF TREATMENT

The compounds of the invention can inhibit cccDNA and have anti-HBV activity. Accordingly, the compounds of the invention are useful for the treatment or prophylaxis of HBV infection.

The invention relates to the use of a compound of formula (I) or (II) for the inhibition of cccDNA.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of Formula (I) or (II), or a pharmaceutically acceptable salts thereof.

### EXAMPLES

The invention will be more fully understood by reference to the following examples. They should not, however, be construed as limiting the scope of the invention.

Abbreviations used herein are as follows:
ACN: acetonitrile
BBr₃: boron tribromide
DMAP: 4-dimethylaminopyridine

### DMF: N,N-dimethylformamide

IC₅₀: the molar concentration of an inhibitor, which produces 50% of the maximum possible response for that inhibitor.
FBS: fetal bovine serum
H₂O₂: hydrogen peroxide
HPLC: high performance liquid chromatography
MS (ESI): mass spectroscopy (electron spray ionization)
Ms: methylsulfonyl
obsd.: observed
PE: petroleum ether
EtOAc: ethyl acetate
DCM: dichloromethane
AcOH: acetic acid
THF: tetrahydrofuran
TFA: trifluoroacetic acid
LiAlH₄: lithium aluminium hydride
LiBH₄: lithium boronhydride
TEA: triethyl amine
NMP: N-methyl-2-pyrrolidone
POCl₃: phosphorus(V) oxychloride
BF₃.Et₂O: boron trifluoride etherate
S-Phos: 2-dicyclohexylphosphino-2′,6′-dimethoxybiphenyl
Ts: p-tolylsulfonyl
δ: chemical shift

### GENERAL EXPERIMENTAL CONDITIONS

Intermediates and final compounds were purified by flash chromatography using one of the following instruments: i) Biotage SP1 system and the Quad 12/25 Cartridge module. ii) column chromatography on silica gel combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200−300 or 300-400.

Intermediates and final compounds were purified by preparative HPLC on reversed phase column using X Bridge™ Perp C₁₈ (5 μm, OBD^{TM} 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):

Acidic condition: A: 0.1% formic acid and 1% acetonitrile in H₂O; B: 0.1% formic acid in acetonitrile; Basic condition: A: 0.05% NH₃·H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Example 1: 2-(4-bromophenyl)-8-chloro-3,4-dihydroquinazoline-4-carboxylic Acid

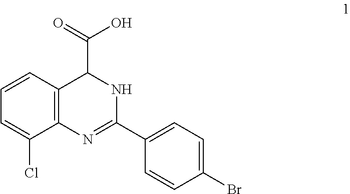 [SMILES: O=C(O)C1NC(c2ccc(Br)cc2)=Nc2c(Cl)cccc21]

Step 1: Preparation of 1-(4-bromobenzoyl)-7-chloro-indoline-2,3-dione

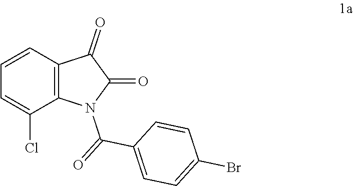 [SMILES: O=C1C(=O)N(C(=O)c2ccc(Br)cc2)c2c(Cl)cccc21]

To a solution of 4-bromobenzoyl chloride (6.65 g, 30.29 mmol) in pyridine (30.0 mL, 370.92 mmol) were added 7-chloroisatin (5.0 g, 27.54 mmol) at 0° C., then the mixture was stirred at 90° C. for 2 hours. The mixture was adjusted to pH˜7 by addition of 1N HCl solution and the resulting mixture was extracted with EtOAc (100 mL) three times. The combined organic layer was washed with brine (100 mL) twice, dried over Na₂SO₄, and concentrated in vacuo to give the crude of 1-(4-bromobenzoyl)-7-chloro-indoline-2,3-dione (8.0 g, 79.69% yield) as a brown solid, which was used in the next step directly without further purification. MS obsd. (ESI⁺) [(M+H₂O+H)⁺]:383.9.

Step 2: Preparation of 2-(4-bromophenyl)-8-chloro-quinazoline-4-carboxylic Acid

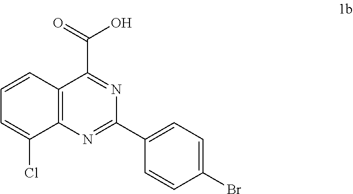 [SMILES: O=C(O)c1nc(-c2ccc(Br)cc2)nc2c(Cl)cccc12]

A mixture of 1-(4-bromobenzoyl)-7-chloro-indoline-2,3-dione (1.0 g, 2.74 mmol) and ammonium acetate (2.1 g, 27.4 mmol) in ethanol (20 mL) was stirred at 90° C. under microwave condition for 30 minutes. The mixture was then concentrated in vacuo and the residue was suspended in water (40 mL). The mixture was then adjusted to pH˜7 by addition of 1N HCl solution. The resulting mixture was extracted with EtOAc (60 mL) three times, the combined organic layer was dried over Na₂SO₄, concentrated in vacuo. The residue was purified by column chromatography on silica gel (elution with DCM:MeOH 10:1 to 2:1) to give 2-(4-bromophenyl)-8-chloro-quinazoline-4-carboxylic acid (260 mg, 26.1% yield). MS obsd. (ESI⁺) [(M+H)⁺]:363.1.

Step 3: Preparation of 2-(4-bromophenyl)-8-chloro-3,4-dihydroquinazoline-4-carboxylic Acid

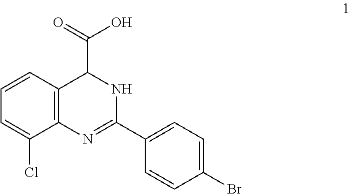 [SMILES: O=C(O)C1NC(c2ccc(Br)cc2)=Nc2c(Cl)cccc21]

To a solution of 2-(4-bromophenyl)-8-chloro-quinazoline-4-carboxylic acid (150 mg, 0.41 mmol) in methanol (5 mL) was added NaBH₄ (418 mg, 12.4 mmol) in ice-water bath. The mixture was then stirred at room temperature for 2 hours. After the reaction was completed, the reaction mixture was then adjusted to pH˜7 by addition of AcOH. The mixture was then concentrated in vacuo and the residue was purified by preparative HPLC to give 2-(4-bromophenyl)-8-chloro-3,4-dihydroquinazoline-4-carboxylic acid (7 mg, 4.4% yield) as an off-white solid. ¹H NMR (400 MHz, DMSO-d6): δ ppm 12.97 (s, 1H), 8.55 (s, 1H), 7.99 (d, J=8.5 Hz, 2H), 7.76 (d, J=8.6 Hz, 2H), 7.36 (dd, J=7.9, 1.1 Hz, 1H), 7.21 (d, J=7.1 Hz, 1H), 7.04 (t, J=7.7 Hz, 1H), 5.26 (s, 1H). MS obsd. (ESI⁺) [(M+H)⁺]:365.0.

### Example 2: 8-chloro-2-phenyl-3,4-dihydroquinazoline-4-carboxylic Acid

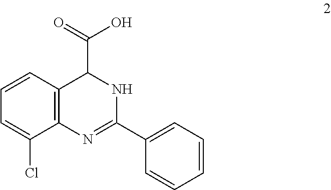 [SMILES: O=C(O)C1NC(c2ccccc2)=Nc2c(Cl)cccc21]

Example 2 was prepared in analogy to the procedure described for the preparation of example 1 by using benzoyl chloride instead of 4-bromobenzoyl chloride in step 1. ¹H NMR (400 MHz, MeOH-d4): δ ppm 7.82-7.92 (m, 2H), 7.67-7.75 (m, 1H), 7.50-7.61 (m, 2H), 7.31-7.42 (m, 2H), 7.13-7.23 (m, 1H). MS obsd. (ESI⁺) [(M+H)⁺]:287.2.

### Example 3: 8-chloro-2-(4-chlorophenyl)-3,4-dihydroquinazoline-4-carboxylic Acid

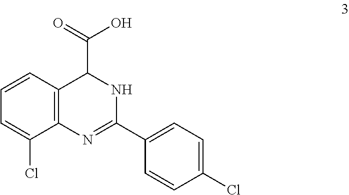 [SMILES: O=C(O)C1NC(c2ccc(Cl)cc2)=Nc2c(Cl)cccc21]

Example 3 was prepared in analogy to the procedure described for the preparation of example 1 by using benzoyl chloride instead of 4-bromobenzoyl chloride in step 1. ¹H NMR (400 MHz, DMSO-d6): δ ppm 7.78-7.94 (m, 2H), 7.57-7.69 (m, 2H), 7.32-7.49 (m, 2H), 7.13-7.34 (m, 1H), 5.43-5.68 (m, 1H). MS obsd. (ESI⁺) [(M+H)⁺]:321.1.

### Example 4: 8-chloro-2-(4-methoxyphenyl)-3,4-dihydroquinazoline-4-carboxylic Acid

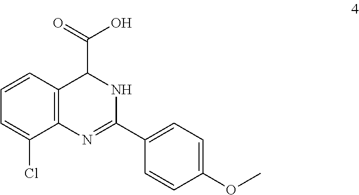 [SMILES: COc1ccc(C2=Nc3c(Cl)cccc3C(C(=O)O)N2)cc1]

Example 4 was prepared in analogy to the procedure described for the preparation of example 1 by using 4-methoxybenzoyl chloride instead of 4-bromobenzoyl chloride in step 1. ¹H NMR (400 MHz, DMSO-d6): δ ppm 8.34-8.46 (m, 2H), 7.91-8.06 (m, 2H), 7.50 (br dd, J=12.7, 8.0 Hz, 2H), 7.28-7.37 (m, 1H), 7.23 (br d, J=8.7 Hz, 2H), 3.96 (s, 3H). MS obsd. (ESI⁺) [(M+H)⁺]:317.1.

### Example 5: 2-(4-bromo-3-methyl-phenyl)-8-chloro-3,4-dihydroquinazoline-4-carboxylic Acid

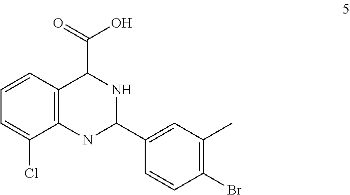 [SMILES: Cc1cc(C2Nc3c(Cl)cccc3C(C(=O)O)N2)ccc1Br]

Example 5 was prepared in analogy to the procedure described for the preparation of example 1 by using 4-bromo-3-methyl-benzoyl chloride instead of 4-bromobenzoyl chloride in step 1. ¹H NMR (400 MHz, DMSO-d6): δ ppm 7.90-8.06 (m, 1H), 7.70-7.86 (m, 2H), 7.39-7.49 (m, 1H), 7.28 (br s, 1H), 7.13 (br dd, J=3.7, 2.3 Hz, 1H), 5.33-5.49 (m, 1H), 2.46 (s, 3H). MS obsd. (ESI⁺) [(M+H)⁺]:379.4.

### Example 6: 2-(3-bromophenyl)-7-(trifluoromethyl)-3,4-dihydroquinazoline-4-carboxylic Acid

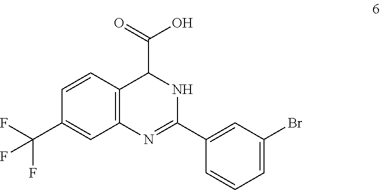 [SMILES: O=C(O)C1NC(c2cccc(Br)c2)=Nc2cc(C(F)(F)F)ccc21]

Example 6 was prepared in analogy to the procedure described for the preparation of example 1 by using 6-(trifluoromethyl)indoline-2,3-dione and 3-bromobenzoyl chloride instead of 7-chloroisatin and 4-bromobenzoyl chloride in step 1. ¹H NMR (400 MHz, DMSO-d6): δ ppm 8.12-8.23 (m, 1H), 7.99-8.04 (m, 1H), 7.76-7.80 (m, 1H), 7.45-7.55 (m, 2H), 7.36-7.44 (m, 1H), 7.33-7.35 (m, 1H), 5.35-5.39 (m, 1H). MS obsd. (ESI⁺) [(M+H)⁺]:399.3.

### Example 7: 2-(4-bromophenyl)-7-(trifluoromethyl)-3,4-dihydroquinazoline-4-carboxylic Acid

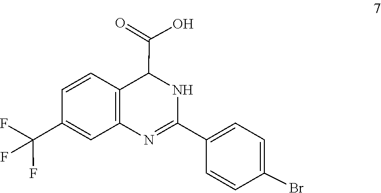 [SMILES: O=C(O)C1NC(c2ccc(Br)cc2)=Nc2cc(C(F)(F)F)ccc21]

Example 7 was prepared in analogy to the procedure described for the preparation of example 1 by using 6-(trifluoromethyl)indoline-2,3-dione instead of 7-chloroisatin chloride in step 1. ¹H NMR (400 MHz, DMSO-d6): δ 7.94-8.00 (m, 2H), 7.70-7.79 (m, 2H), 7.26-7.50 (m, 3H), 5.28-5.38 (m, 1H). MS obsd. (ESI⁺) [(M+H)⁺]:399.1.

Example 8: 8-chloro-2-(6-methoxy-3-pyridyl)-3,4-dihydroquinazoline-4-carboxylic Acid

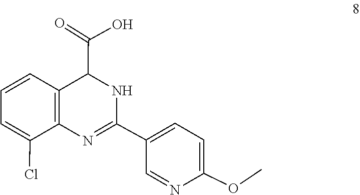 [SMILES: COc1ccc(C2=Nc3c(Cl)cccc3C(C(=O)O)N2)cn1]

Step 1: Preparation of 7-chloro-1-(6-chloropyridine-3-carbonyl)indoline-2,3-dione 8a

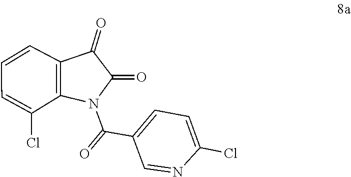 [SMILES: O=C1C(=O)N(C(=O)c2ccc(Cl)nc2)c2c(Cl)cccc21]

Intermediate 8a was prepared in analogy to the procedure described for the preparation of example 1 by using 6-chloropyridine-3-carbonyl chloride instead of 4-bromobenzoyl chloride in step 1.

Step 2: Preparation of ethyl 8-chloro-2-(6-chloro-3-pyridyl)quinazoline-4-carboxylate

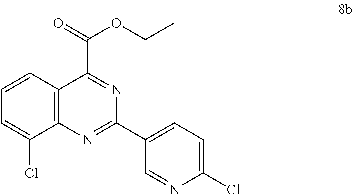 [SMILES: CCOC(=O)c1nc(-c2ccc(Cl)nc2)nc2c(Cl)cccc12]

A mixture of compound 7-chloro-1-(6-chloronicotinoyl)indoline-2,3-dione (1.6 g, 4.98 mmol) and ammonium acetate (3.84 g, 49.8 mmol) in ethanol (100 mL) was stirred at 90° C. under microwave condition for 30 minutes. The mixture was then concentrated in vacuo and the residue suspended in water (40 mL). The mixture was then adjusted to pH˜7 by addition of 1N HCl solution. The resulting mixture was extracted twice with EtOAc (60 mL) three times, the combined organic layer was dried over Na₂SO₄, concentrated in vacuo. The residue was purified by column chromatography on silica gel (elution with DCM:MeOH 10:1 to 2:1) to ethyl 8-chloro-2-(6-chloro-3-pyridyl)quinazoline-4-carboxylate (0.28 g, 16.1% yield) as a white solid.

Step 3: Preparation of 8-chloro-2-(6-methoxy-3-pyridyl)quinazoline-4-carboxylic Acid

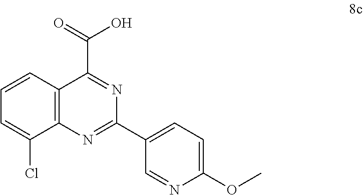 [SMILES: COc1ccc(-c2nc(C(=O)O)c3cccc(Cl)c3n2)cn1]

A mixture of ethyl 8-chloro-2-(6-chloro-3-pyridyl)quinazoline-4-carboxylate (0.25 g, 718 μmol) and sodium methoxide (194 mg, 3.59 mmol) in MeOH (20 mL) was stirred at 75° C. for 16 hours. The mixture was then concentrated in vacuo and the residue suspended in water (20 mL). The mixture was then adjusted to pH˜7 by addition of 1N HCl solution. The resulting mixture was extracted twice with EtOAc (30 mL) three times, the combined organic layer was dried over Na₂SO₄, concentrated in vacuo. The residue was used in the next step directly without further purification. MS obsd. (ESI⁺) [(M+H)⁺]:316.1.

Step 4: Preparation of 8-chloro-2-(6-methoxy-3-pyridyl)-3,4-dihydroquinazoline-4-carboxylic Acid

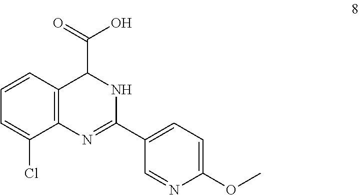 [SMILES: COc1ccc(C2=Nc3c(Cl)cccc3C(C(=O)O)N2)cn1]

To a solution of 8-chloro-2-(6-methoxy-3-pyridyl)quinazoline-4-carboxylic acid (150 mg, 475 μmol) in MeOH (50 mL) was added LiBH₄ (1.03 g, 47.5 mmol) at room temperature. The mixture was then stirred room temperature for 72 hours. The mixture was then adjusted to pH˜7 by addition of AcOH and the resulting mixture was then concentrated in vacuo. The residue was then purified by preparative HPLC to give 8-chloro-2-(6-methoxy-3-pyridyl)-3,4-dihydroquinazoline-4-carboxylic acid (7 mg, 4.4% yield) as a white solid. ¹H NMR (400 MHz, DMSO-d6): δ ppm 12.95-13.12 (m, 1H), 8.82 (d, J=2.0 Hz, 1H), 8.54 (br d, J=2.2 Hz, 1H), 8.28-8.36 (m, 1H), 7.31-7.45 (m, 1H), 7.17-7.25 (m, 1H), 6.91-7.07 (m, 2H), 5.19-5.32 (m, 1H), 3.88-3.97 (s, 3H). MS obsd. (ESI⁺) [(M+H)⁺]:318.1.

### Example 9: 8-chloro-2-(6-ethoxy-3-pyridyl)-3,4-dihydroquinazoline-4-carboxylic Acid

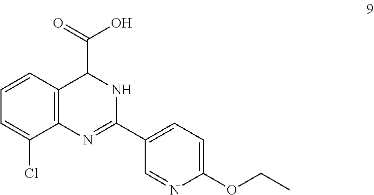 [SMILES: CCOc1ccc(C2=Nc3c(Cl)cccc3C(C(=O)O)N2)cn1]

Example 9 was prepared in analogy to the procedure described for the preparation of example 8 by using sodium ethanolate instead of sodium methoxide in step 3. ¹H NMR (400 MHz, DMSO-d6): δ 8.71-8.88 (m, 1H), 8.28 (br d, J=8.6 Hz, 1H), 7.35-7.47 (m, 1H), 7.20-7.32 (m, 1H), 7.03-7.17 (m, 1H), 6.90-7.03 (m, 1H), 5.19-5.43 (m, 1H), 4.35-4.49 (m, 2H), 1.35 ppm (t, J=7.0 Hz, 3H). MS obsd. (ESI⁺) [(M+H)⁺]:332.1.

### Example 10: 8-chloro-2-(6-pyrrolidin-1-yl-3-pyridyl)-3,4-dihydroquinazoline-4-carboxylic Acid

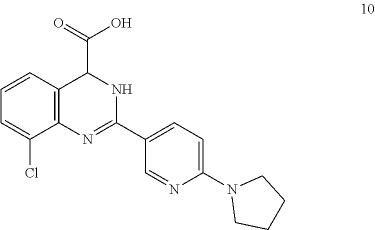 [SMILES: O=C(O)C1NC(c2ccc(N3CCCC3)nc2)=Nc2c(Cl)cccc21]

Example 10 was prepared in analogy to the procedure described for the preparation of example 8 by using pyrrolidine instead of sodium methoxide in step 3. ¹H NMR (400 MHz, DMSO-d6): δ 11.79-12.03 (m, 1H), 10.91-11.15 (m, 1H), 8.53-8.73 (m, 1H), 7.91 (br d, J=8.8 Hz, 1H), 7.56-7.66 (m, 1H), 7.31-7.53 (m, 2H), 6.68-6.80 (m, 1H), 5.65-5.83 (m, 1H), 1.91-2.07 (m, 4H). MS obsd. (ESI⁺) [(M+H)⁺]:357.2.

### Example 11: 2-(4-bromophenyl)-8-chloro-4-methyl-3H-quinazoline-4-carboxylic Acid

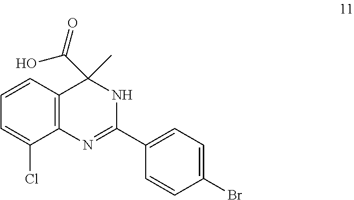 [SMILES: CC1(C(=O)O)NC(c2ccc(Br)cc2)=Nc2c(Cl)cccc21]

To a solution of 2-(4-bromophenyl)-8-chloroquinazoline-4-carboxylic acid (50 mg, 138 μmol) in THF (10 mL) cooled at ice-bath was added MeMgBr (688 μl, 1 mol/L, 688 μmol) dropwise. The mixture was then stirred at room temperature for 2 hours. The mixture was then quenched with saturated NH₄Cl solution and extracted by EtOAc (100 mL). The organic layer was then washed with brine, dried over Na₂SO₄, concentrated in vacuo. The residue was then purified by preparative HPLC to give 2-(4-bromophenyl)-8-chloro-4-methyl-3H-quinazoline-4-carboxylic a cid (14 mg, 26.3% yield) as a white solid. ¹H NMR (400 MHz, DMSO-d6): δ 8.04-8.27 (m, 1H), 7.91-8.04 (m, 2H), 7.71-7.85 (m, 2H), 7.43 (br s, 2H), 6.95-7.14 (m, 1H), 1.73-2.01 ppm (m, 3H). MS obsd. (ESI⁺) [(M+H)⁺]:379.0.

### Example 12: methyl 2-(4-bromophenyl)-8-chloro-3,4-dihydroquinazoline-4-carboxylate

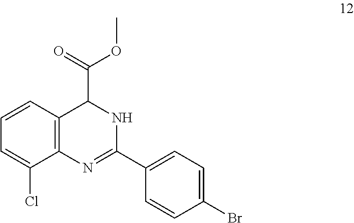 [SMILES: COC(=O)C1NC(c2ccc(Br)cc2)=Nc2c(Cl)cccc21]

To a mixture of 2-(4-bromophenyl)-8-chloro-3,4-dihydroquinazoline-4-carboxylic acid (200 mg, 547 μmol) and K₂CO₃ (227 mg, 1.64 mmol) in DMF (5 mL) was added iodomethane (155 mg, 1.09 mmol) and the reaction mixture was stirred at 80° C. for 1 hour. After the reaction was completed, the mixture was diluted with EtOAc (100 mL). The mixture was then filtred and the filtrate was concentrated in vacuo. The residue was then purified by preparative HPLC to give methyl 2-(4-bromophenyl)-8-chloro-3,4-dihydroquinazoline-4-carboxylate (6 mg, 2.83% yield) as a white solid. ¹H NMR (400 MHz, DMSO-d6): δ 7.88-7.99 (m, 2H), 7.68-7.75 (m, 2H), 7.37-7.42 (m, 1H), 7.26 (d, J=7.5 Hz, 1H), 7.02-7.14 (m, 1H), 5.37-5.42 (m, 1H), 3.73 (s, 3H). MS obsd. (ESI⁺) [(M+H)⁺]:377.8.

### Example 13: 2-(4-bromophenyl)-8-chloro-3,4-dihydroquinazoline-4-carboxamide

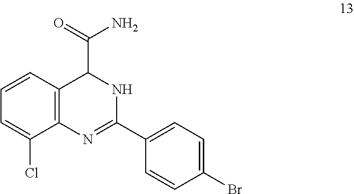 [SMILES: NC(=O)C1NC(c2ccc(Br)cc2)=Nc2c(Cl)cccc21]

Step 1: Preparation of 2-(4-bromophenyl)-8-chloro-quinazoline-4-carboxamide

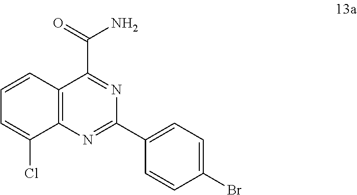 [SMILES: NC(=O)c1nc(-c2ccc(Br)cc2)nc2c(Cl)cccc12]

To a solution of 4-bromobenzoyl chloride (6.65 g, 30.29 mmol) in pyridine (30.0 mL, 370.92 mmol) were added 7-chloroisatin (5.0 g, 27.54 mmol) at ice-water condition, then the mixture was stirred at 90° C. for 2 h. The mixture was adjusted to pH˜7 by addition of 1N HCl solution and the resulting mixture was extracted with EtOAc (100 mL) three times. The combined organic layer was washed with brine (100 mL) twice, dried over Na₂SO₄, filtered and concentrated in vacuo to give the crude of 1-(4-bromobenzoyl)-7-chloro-indoline-2,3-dione (8 g, 79.69% yield) as a brown solid, which was used for next step directly without further purification. MS obsd. (ESI⁺) [(M+H₂O+H)⁺]:383.9.

A mixture of 1-(4-bromobenzoyl)-7-chloro-indoline-2,3-dione (1 g, 2.74 mmol) and ammonium acetate (2.11 g, 27.4 mmol) in ethanol (20 mL) was stirred at 90° C. under microwave condition for 30 minutes. The mixture was then concentrated in vacuo and the residue suspended in water (40 mL). The mixture was then adjusted to pH˜7 by addition of 1N HCl solution. The resulting mixture was extracted twice with EtOAc (60 mL) three times, the combined organic layer was dried over Na₂SO₄, concentrated in vacuo. The residue was purified by column chromatography on silica gel (elution with DCM:MeOH 10:1 to 2:1) to give 2-(4-bromophenyl)-8-chloro-quinazoline-4-carboxamide (200 mg, 20.1% yield). MS obsd. (ESI⁺) [(M+H)⁺]:361.9.

Step 2: Preparation of 2-(4-bromophenyl)-8-chloro-3,4-dihydroquinazoline-4-carboxamide

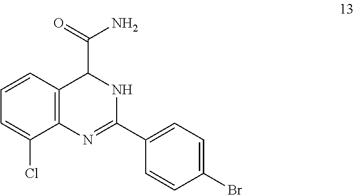 [SMILES: NC(=O)C1NC(c2ccc(Br)cc2)=Nc2c(Cl)cccc21]

To a solution of 2-(4-bromophenyl)-8-chloro-quinazoline-4-carboxamide (100 mg, 276 μmol) in MeOH (10 mL) was added LiBH₄ (120 mg, 5.52 mmol) at room temperature and the mixture was then stirred room temperature for 5 hours. After the reaction was completed, the solution was adjusted to pH˜7 by addition of AcOH. The resulting mixture was concentrated in vacuo and the residue was purified by preparative HPLC to give 2-(4-bromophenyl)-8-chloro-3,4-dihydroquinazoline-4-carboxamide (10 mg, 9.85% yield) as a white solid. ¹H NMR (400 MHz, DMSO-d6): δ 7.81-7.97 (m, 4H), 7.59-7.64 (m, 1H), 7.49-7.55 (m, 1H), 7.38-7.46 (m, 1H), 5.55-5.64 (m, 1H). MS obsd. (ESI⁺) [(M+H)⁺]:363.8.

### Example 14: 8-chloro-2-(4-methoxyphenyl)-3,4-dihydroquinazoline-4-carboxamide

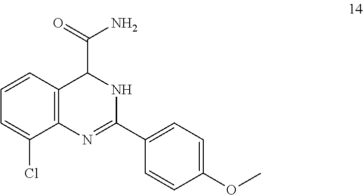 [SMILES: COc1ccc(C2=Nc3c(Cl)cccc3C(C(N)=O)N2)cc1]

Example 14 was prepared in analogy to the procedure described for the preparation of example 13 by using 4-methoxybenzoyl chloride instead of 4-bromobenzoyl chloride in step 1. ¹H NMR (400 MHz, MeOH-d4): δ 8.26-8.31 (m, 1H), 7.77-7.83 (m, 2H), 7.34-7.40 (m, 1H), 7.24-7.31 (m, 1H), 7.10-7.16 (m, 1H), 6.98-7.06 (m, 2H), 5.29-5.33 (m, 1H), 3.77-3.84 (s, 3H). MS obsd. (ESI⁺) [(M+H)⁺]:316.2.

### Example 15: 2-(3-bromophenyl)-7-(trifluoromethyl)-3,4-dihydroquinazoline-4-carboxamide

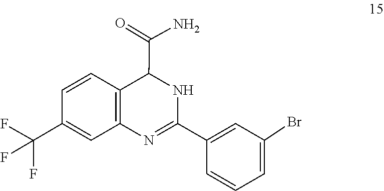 [SMILES: NC(=O)C1NC(c2cccc(Br)c2)=Nc2cc(C(F)(F)F)ccc21]

Example 15 was prepared in analogy to the procedure described for the preparation of example 13 by using 6-(trifluoromethyl)indoline-2,3-dione and 3-bromobenzoyl chloride instead of 7-chloroisatin and 4-bromobenzoyl chloride in step 1. ¹H NMR (400 MHz, MeOH-d4): δ ppm 8.14-8.22 (m, 1H), 8.00-8.05 (m, 1H), 7.90-7.96 (m, 1H), 7.49-7.76 (m, 4H), 5.73 ppm (s, 1H). MS obsd. (ESI⁺) [(M+H)⁺]:398.1.

### Example 16: 8-chloro-N-(2-hydroxyethyl)-2-(4-methoxyphenyl)-3,4-dihydroquinazoline-4-carboxamide

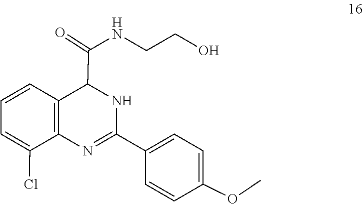 [SMILES: COc1ccc(C2=Nc3c(Cl)cccc3C(C(=O)NCCO)N2)cc1]

Step 1: Preparation of 7-chloro-1-(4-methoxybenzoyl)indoline-2,3-dione

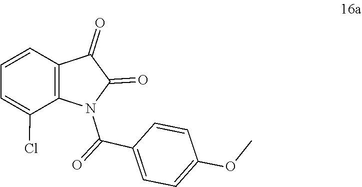 [SMILES: COc1ccc(C(=O)N2C(=O)C(=O)c3cccc(Cl)c32)cc1]

To a mixture of 7-chloroindoline-2,3-dione (1 g, 5.51 mmol) in pyridine (16 mL) were added 4-methoxybenzoyl chloride (1.03 g, 6.06 mmol) at 0° C., then the mixture was stirred at 90° C. for 1 hour. The mixture was adjusted to pH˜7 by addition of 1N HCl solution and the resulting mixture was extracted with EtOAc (30 mL) three times. The combined organic layer was washed with brine (30 mL) twice, dried over Na₂SO₄, filtered and concentrated in vacuo to give the crude of 7-chloro-1-(4-methoxybenzoyl)indoline-2,3-dione (1.6 g, 92% yield) as a brown solid, which was used for next step directly without further purification.

Step 2: Preparation of 8-chloro-2-(4-methoxyphenyl)quinazoline-4-carboxylic Acid

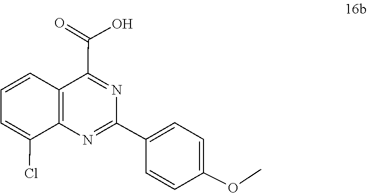 [SMILES: COc1ccc(-c2nc(C(=O)O)c3cccc(Cl)c3n2)cc1]

A mixture of compound 7-chloro-1-(4-methoxybenzoyl)indoline-2,3-dione (1.6 g, 5.07 mmol) and ammonium acetate (3.97 g, 50.7 mmol) in ethanol (20 mL) was stirred at 90° C. under microwave condition for 30 minutes. The mixture was then concentrated in vacuo and the residue suspended in water (40 mL). The mixture was then adjusted to pH˜7 by addition of 1N HCl solution. The resulting mixture was extracted twice with EtOAc (60 mL) three times, the combined organic layer was dried over Na₂SO₄, concentrated in vacuo. The residue was purified by column chromatography on silica gel (elution with DCM:MeOH 10:1 to 2:1) to give 8-chloro-2-(4-methoxyphenyl)quinazoline-4-carboxylic acid (250 mg, 15.7% yield).

Step 3: Preparation of 8-chloro-N-(2-hydroxyethyl)-2-(4-methoxyphenyl)quinazoline-4-carboxamide

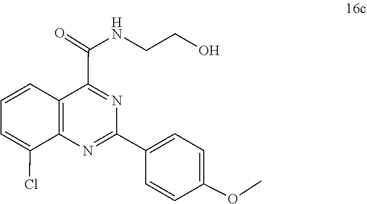 [SMILES: COc1ccc(-c2nc(C(=O)NCCO)c3cccc(Cl)c3n2)cc1]

To a solution of 8-chloro-2-(4-methoxyphenyl)quinazoline-4-carboxylic acid (250 mg, 794 μmol) in DCM (20 mL) was added oxalyl dichloride (106 mg, 71.5 μL, 834 μmol) dropwise. Then 2 drops of DMF was added and the reaction mixture was stirred at room temp for 2 hours. The resulting solution was then added to a mixture of 2-aminoethanol (229 mg, 226 μl, 3.75 mmol) and triethylamine (152 mg, 1.5 mmol) in DCM (10 mL) at room temperature. After addition, the mixture was stirred at room temperature for 30 minutes. The mixture was quenched with water (20 mL) and extracted with EtOAc (30 mL) three times. The combined organic layer was washed with brine, dried over anhydrous Na₂SO₄ and concentrated in vacuo to give the crude of 8-chloro-N-(2-hydroxyethyl)-2-(4-methoxyphenyl)quinazoline-4-carboxamide (250 mg, 91.3% yield) as a yellow solid, which was used in the next step directly without further purification. MS obsd. (ESI⁺) [(M+H)⁺]:358.2.

Step 4: Preparation of 8-chloro-N-(2-hydroxyethyl)-2-(4-methoxyphenyl)-3,4-dihydroquinazoline-4-carboxamide

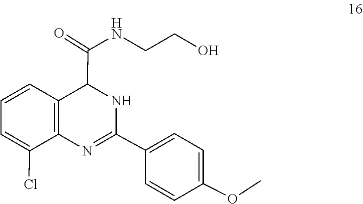 [SMILES: COc1ccc(C2=Nc3c(Cl)cccc3C(C(=O)NCCO)N2)cc1]

To a solution of 8-chloro-N-(2-hydroxyethyl)-2-(4-methoxyphenyl)quinazoline-4-carboxamide (50 mg, 140 μmol) in a mixed solvent of MeOH (5 mL) and THF (5 mL) was added LiBH₄ (60.9 mg, 2.79 mmol) at room temperature. The mixture was stirred room temperature for 1 hour. After the reaction was completed, the reaction was quenched by addition of AcOH (720 mg, 12mmol) and the mixture was concentrated in vacuo. The residue was then purified by preparative HPLC to give 8-chloro-N-(2-hydroxyethyl)-2-(4-methoxyphenyl)-3,4-dihydroquinazoline-4-carboxamide (34 mg, 64.2% yield) as a white powder. ¹H NMR (400 MHz, MeOH-d4): δ ppm 8.60-8.82 (m, 1H), 7.77-7.90 (m, 1H), 7.49-7.62 (m, 1H), 7.18-7.42 (m, 2H), 5.49-5.64 (m, 1H), 3.50-3.79 (m, 3H), 3.33-3.50 (m, 2H), 3.02-3.35 ppm (m, 1H). MS obsd. (ESI⁺) [(M+H)⁺]:360.2.

### Example 17: [2-(4-bromophenyl)-7-(trifluoromethyl)-3,4-dihydroquinazolin-4-yl]-morpholino-methanone

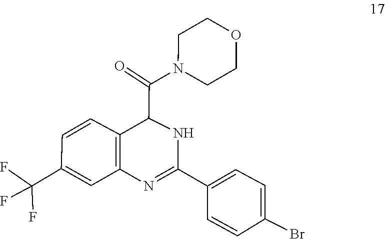 [SMILES: O=C(C1NC(c2ccc(Br)cc2)=Nc2cc(C(F)(F)F)ccc21)N1CCOCC1]

Example 17 was prepared in analogy to the procedure described for the preparation of example 16 by using 6-(trifluoromethyl)indoline-2,3-dione, 4-bromobenzoyl chloride instead of 7-chloroisatin, 4-methoxybenzoyl chloride in step 1 and using morpholine instead of 2-aminoethanol in step 3. ¹H NMR (400 MHz, MeOH-d4): δ ppm 7.81-8.00 (m, 4H), 7.63-7.72 (m, 1H), 7.48-7.59 (m, 2H), 6.25-6.43 (m, 1H), 3.99 (br s, 2H), 3.47-3.92 (m, 6H). MS obsd. (ESI⁺) [(M+H)⁺]:468.1.

### Example 18: 2-(4-bromophenyl)-8-chloro-N-(2-chloroethyl)-3,4-dihydroquinazoline-4-carboxamide

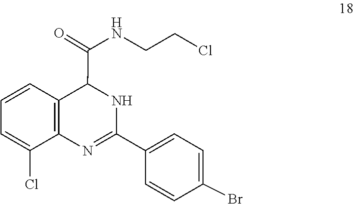 [SMILES: O=C(NCCCl)C1NC(c2ccc(Br)cc2)=Nc2c(Cl)cccc21]

Example 18 was prepared in analogy to the procedure described for the preparation of example 16 by using 4-bromobenzoyl chloride instead of 4-methoxybenzoyl chloride in step 1 and using 2-chloroethanamine hydrochloride instead of 2-aminoethanol in step 3. ¹H NMR (400 MHz, MeOH-d4): δ ppm 7.82-7.93 (m, 4H), 7.55-7.64 (m, 1H), 7.43-7.52 (m, 1H), 7.37-7.44 (m, 1H), 5.53-5.65 (m, 1H), 3.51-3.75 ppm (m, 4H). MS obsd. (ESI⁺) [(M+H)⁺]:424.0.

### Example 19: 3-[2-[4-[4-methyl-7-(trifluoromethyl)-3,4-dihydroquinazolin-2-yl]phenoxy]ethoxy]cyclobutanecarboxylic Acid

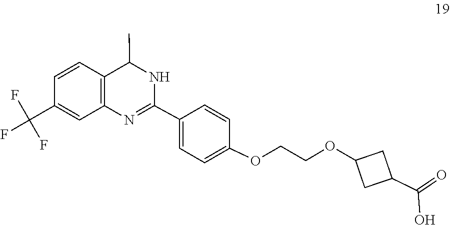 [SMILES: CC1NC(c2ccc(OCCOC3CC(C(=O)O)C3)cc2)=Nc2cc(C(F)(F)F)ccc21]

Step 1: Preparation of 2-benzyloxyethoxy(trimethyl)silane

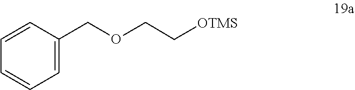

To a solution of 2-benzyloxyethanol (20.0 g, 131.4 mmol) and TEA (20.0 g, 197.1 mmol) in dichloromethane (200 mL) cooled at 0° C. was added trimethylsilyl chloride (17.1 g, 157.7 mmol) and the mixture was then stirred at 25° C. for 16 hours. After the reaction was completed, the mixture was concentrated in vacuo and the residue was purified by column chromatography on silica gel (eluted with PE:EtOAc=50:1 to 10:1) to give the 2-benzyloxyethoxy(trimethyl)silane (25.0 g, 84.9% yield) as colorless oil.

Step 2: Preparation of methyl 3-(2-benzyloxyethoxy)cyclobutanecarboxylate

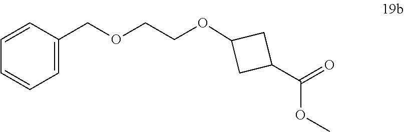 [SMILES: COC(=O)C1CC(OCCOCc2ccccc2)C1]

To a solution of 2-benzyloxyethoxy(trimethyl)silane (25.0 g, 111.4 mmol) and methyl 3-oxocyclobutanecarboxylate (CAS #: 4934-99-0, Cat.#: PB01390, from PharmaBlock (NanJing) R&D Co. Ltd,15.0 g, 117.0 mmol) in dichloromethane (200 mL) was added trimethylsilyl trifluoromethanesulfonate (12.4 g, 55.7 mmol) dropwise at −78° C. After addition, the mixture was stirred at −78° C. for additional 1 hour, then to the resulting mixture was added triethylsilane (14.25 g, 122.57 mmol) at −78° C. The resulting mixture was then warmed to room temperature and stirred for additional 1 hour. After the reaction was completed, the mixture was washed with saturated NH₄Cl solution, brine, dried over anhydrous sodium sulfate, and concentrated in vacuo. The residue was purified by column chromatography on silica gel (elution with PE/EtOAc=100:1˜50:1) to give methyl 3-(2-benzyloxyethoxy)cyclobutanecarboxylate (28 g, 95.1% yield) as colorless oil. MS obsd. (ESI⁺) [(M+H)⁺]:265.1.

Step 3: Preparation of methyl 3-(2-hydroxyethoxy)cyclobutanecarboxylate

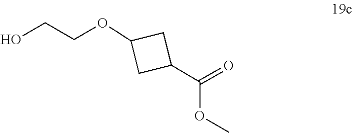

To a solution of methyl 3-(2-benzyloxyethoxy)cyclobutanecarboxylate (28.0 g, 105.9 mmol) in MeOH (300.0 mL) was added wet Pd(OH)₂ (1.48 g, 10.6 mmol) at room temperature and the mixture was then hydrogenated under H₂ atmosphere at room temperature overnight. After the reaction was completed, the reaction was filtered through silica gel pad and the filtrate was concentrated in vacuo to give the crude methyl 3-(2-hydroxyethoxy)cyclobutanecarboxylate (18 g, 97.6% yield) as colorless oil.

Step 4: Preparation of methyl 3-[2-(p-tolylsulfonyloxy)ethoxy]cyclobutanecarboxylate

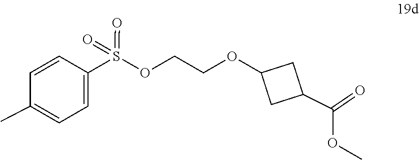

To a solution of methyl 3-(2-hydroxyethoxy)cyclobutanecarboxylate (5 g, 28.7 mmol) and DMAP (5.26 g, 43.1 mmol) in dichloromethane (80 mL) was added 4-methylbenzene-1-sulfonyl chloride (6.02 g, 31.6 mmol) at room temperature and the mixture was then stirred at room temperature overnight. After the reaction was completed, the mixture was washed with 1N HC1 (25 mL), water (15mL), saturated NaHCO₃ solution, brine and concentrated in vacuo to give the crude methyl 3-[2-(p-tolylsulfonyloxy)ethoxy]cyclobutanecarboxylate (8.1 g, 85.6% yield) as colorless oil, which was used in next step directly without further purification. MS obsd. (ESI⁺) [(M+H)⁺]:329.2.

Step 5: Preparation of 2-(4-methoxyphenyl)-4-methyl-7-(trifluoromethyl)quinazoline

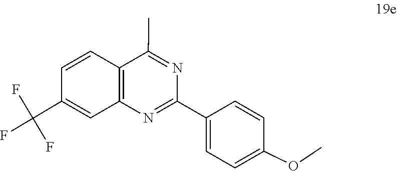 [SMILES: COc1ccc(-c2nc(C)c3ccc(C(F)(F)F)cc3n2)cc1]

To a solution of 1-[2-amino-4-(trifluoromethyl)phenyl]ethanone (4.0 g, 19.69 mmol) in ACN (150 mL) was added 4-methoxybenzaldehyde (6061.72 mg, 49.22 mmol), tert-Butyl hydroperoxide (17743.65 mg, 137.82 mmol) and ammonium cerium(IV) nitrate (864.79 mg, 1.97 mmol). The mixture was stirred at 80° C. for 14 hours. The mixture was diluted with EtOAc (100 mL) and the resulting suspension was filtered. The filtrate was then concentrated in vacuo and the residue was purified by column chromatography on silica gel (elution with PE:EtOAc 100:1 to 2:1) to give 2-(4-methoxyphenyl)-4-methyl-7-(trifluoromethyl)quinazoline (3 g, 46.91% yield) as a light yellow solid. MS obsd. (ESI⁺) [(M+H)⁺]:319.1

Step 6: Preparation of 4-[4-methyl-7-(trifluoromethyl)quinazolin-2-yl]phenol

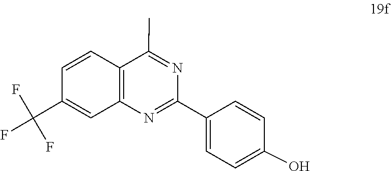 [SMILES: Cc1nc(-c2ccc(O)cc2)nc2cc(C(F)(F)F)ccc12]

To a solution of 2-(4-methoxyphenyl)-4-methyl-7-(trifluoromethyl)quinazoline (3.0 g, 9.43 mmol) in DCM (50 mL) was added boron tribromide (9.45 g, 37.7 mmol). The mixture was stirred at 25° C. for 1 hour. After the reaction was completed, the mixture was quenched with ice-water (30 mL) and extracted with EtOAc (30 mL) three times. The combined organic layer was washed with brine, dried over anhydrous sodium sulfate, and concentrated in vacuo. The residue was purified by column chromatography on silica gel (elution with PE:EtOAc=5:1˜1:1) to give 4-[4-methyl-7-(trifluoromethyl)quinazolin-2-yl]phenol (750 mg, 25.11% yield) as a light yellow solid. MS obsd. (ESI⁺) [(M+H)⁺]:305.2.

Step 7: Preparation of 4-[4-methyl-7-(trifluoromethyl)-3,4-dihydroquinazolin-2-yl]phenol

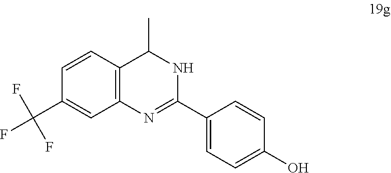 [SMILES: CC1NC(c2ccc(O)cc2)=Nc2cc(C(F)(F)F)ccc21]

To a solution of 4-[4-methyl-7-(trifluoromethyl)quinazolin-2-yl]phenol (700.0 mg, 2.3 mmol) in THF (10 mL)/ethanol (10 mL) was added sodium borohydride (3480.36 mg, 92 mmol) in portions at 25° C. The mixture was stirred at 60° C. for 48 hours. Then the mixture was quenched by water (30 mL) and extracted with EtOAc (30 mL) three times. The combined organic layer was washed with brine, dried over anhydrous sodium sulfate, and concentrated in vacuo. The residue was then purified by column chromatography on silica gel (elution with PE:EtOAc=5:1˜1:1) to give 4-[4-methyl-7-(trifluoromethyl)-3,4-dihydroquinazolin-2-yl]phenol (700 mg, 100% yield) as a white solid. MS obsd. (ESI⁺) [(M+H)⁺]:307.2.

Step 8: Preparation of tert-butyl 2-(4-hydroxyphenyl)-4-methyl-7-(trifluoromethyl)-4H-quinazoline-3-carboxylate

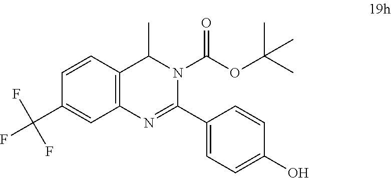 [SMILES: CC1c2ccc(C(F)(F)F)cc2N=C(c2ccc(O)cc2)N1C(=O)OC(C)(C)C]

To a solution of 4-[4-methyl-7-(trifluoromethyl)-3,4-dihydroquinazolin-2-yl]phenol (700 mg, 2.3 mmol) and triethylamine (0.3 mL, 2.12 mmol) in THF (10 mL) was added di-t-butyldicarbonate (1157.95 mg, 5.31 mmol). The mixture was stirred at 25° C. for 4 hours. After the reaction was completed, the mixture was then concentrated in vacuo to give the crude of tert-butyl 2-(4-hydroxyphenyl)-4-methyl-7-(trifluoromethyl)-4H-quinazoline-3-carboxylate (710 mg, 76.44% yield) as a white solid, which was used in the next step directly without further purification. MS obsd. (ESI⁺) [(M+H)⁺]:407.0.

Step 9: Preparation of methyl 3-[2-[4-[4-methyl-7-(trifluoromethyl)-3,4-dihydroquinazolin-2-yl]phenoxy]ethoxy]cyclobutanecarboxylate

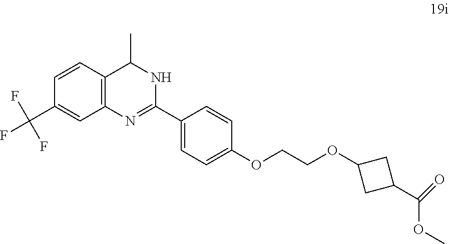 [SMILES: COC(=O)C1CC(OCCOc2ccc(C3=Nc4cc(C(F)(F)F)ccc4C(C)N3)cc2)C1]

To a solution of tert-butyl 2-(4-hydroxyphenyl)-4-methyl-7-(trifluoromethyl)-4H-quinazoline-3-carboxylate (121.2 mg, 0.370 mmol) and K₂CO₃ (153.03 mg, 1.11 mmol) in ACN (2 mL)/methanol (2 mL) was added methyl 3-[2-(p-tolylsulfonyloxy)ethoxy]cyclobutanecarboxylate (150.0 mg, 0.370 mmol). The mixture was stirred at 60° C. for 6 hours. After the reaction was completed, the mixture was then concentrated in vacuo to give the crude of methyl 3-[2-[4-[4-methyl-7-(trifluoromethyl)-3,4-dihydroquinazolin-2-yl]phenoxy]ethoxy]cyclobutanecarboxylate (180 mg, 0.390 mmol, 105.45% yield) as a white solid, which was used in the next step directly without further purification. MS obsd. (ESI⁺) [(M+H)⁺]:462.0.

Step 10: Preparation of 3-[2-[4-[4-methyl-7-(trifluoromethyl)-3,4-dihydroquinazolin-2-yl]phenoxy]ethoxy]cyclobutanecarboxylic Acid

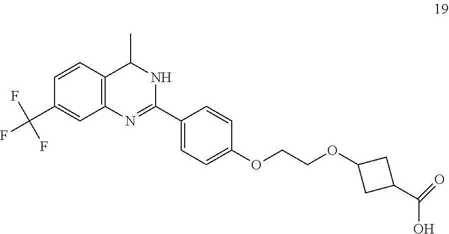 [SMILES: CC1NC(c2ccc(OCCOC3CC(C(=O)O)C3)cc2)=Nc2cc(C(F)(F)F)ccc21]

To a solution of methyl 3-[2-[4-[4-methyl-7-(trifluoromethyl)-3,4-dihydroquinazolin-2-yl]phenoxy]ethoxy]cyclobutanecarboxylate (180.0 mg, 0.390 mmol) in THF (3 mL)/water (3 mL) was added LiOH (0.01 mL, 1.17 mmol). The mixture was stirred at room temperature for 4 hours. The mixture was then adjusted to pH-6 by addition of 6M HCl and extracted with EtOAc (20 mL) three times. The combined organic layer was washed with brine, dried over anhydrous Na₂SO₄ and concentrated in vacuo. The residue was purified by to give 3-[2-[4-[4-methyl-7-(trifluoromethyl)-3,4-dihydroquinazolin-2-yl]phenoxy]ethoxy]cyclobutanecarboxylic acid (30 mg,17.2% yield) as a white solid. ¹H NMR (400 MHz, MeOH-d4): δ ppm 12.17 (br s, 1H), 10.75 (br s, 1H), 7.96-7.86 (m, 1H), 7.92 (d, J=8.9 Hz, 1H), 7.72-7.66 (m, 1H), 7.61 (d, J=9.4 Hz, 2H), 7.29 (d, J=8.4 Hz, 2H), 5.24 (q, J=6.1 Hz, 1H), 4.28-4.21 (m, 2H), 3.95 (td, J=7.4, 14.6 Hz, 1H), 3.74-3.64 (m, 2H), 2.98-2.88 (m, 1H), 2.63-2.59 (m, 1H), 2.47-2.36 (m, 2H), 2.23-2.09 (m, 1H), 2.06-1.92 (m, 1H), 1.62 (d, J=6.6 Hz, 3H). MS obsd. (ESI⁺) [(M+H)⁺]:449.2.

### Example 20: 2-[2-[4-[4-methyl-7-(trifluoromethyl)-3,4-dihydroquinazolin-2-yl]phenoxy]ethoxy]acetic Acid

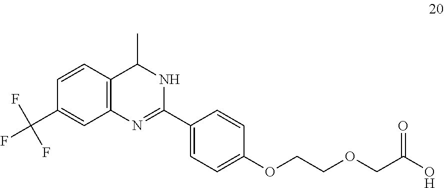 [SMILES: CC1NC(c2ccc(OCCOCC(=O)O)cc2)=Nc2cc(C(F)(F)F)ccc21]

Step 1: Preparation of methyl 2-(2-benzyloxyethoxy)acetate

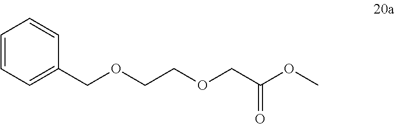 [SMILES: COC(=O)COCCOCc1ccccc1]

To a mixture of NaOH (10M, 300.0 mL), methyl 2-bromoacetate (23.5 g, 120.3 mmol) and tetrabutylammonium iodide (8.8 g, 24.06 mmol) in DCM (300 mL) was added 3-benzyloxypropan-1-ol (12.99 mL, 120.32 mmol) at 30° C. and the mixture was stirred at 30° C. for 72 hours. After the reaction was completed, the organic phase was separated out and the aqualic phase was extracted with DCM (150 mL) twice. The combined organic layer was washed with brine, dried over MgSO₄ and concentrated in vacuo. The residue was purified by column chromatography on silica gel (eluent with PE:EtOAc=3:1) to give methyl 2-(2-benzyloxyethoxy)acetate (21.3 g, 63.3% yield) as a colorless liquid. MS obsd. (ESI⁺) [(M+H)⁺]: 224.1.

Step 2: Preparation of methyl 2-[2-(p-tolylsulfonyloxy)ethoxy]acetate

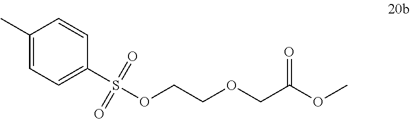

Compound 20b was prepared in analogy to the procedure described for the preparation of compound 19d by using methyl 2-(2-benzyloxyethoxy)acetate as the starting material instead of methyl 3-(2-benzyloxyethoxy)cyclobutanecarboxylate in Step 3. MS obsd. (ESI⁺) [(M+H)⁺]: 289.1.

Step 3: Preparation of 2-[2-[4-[4-methyl-7-(trifluoromethyl)-3,4-dihydroquinazolin-2-yl]phenoxy]ethoxy]acetic Acid

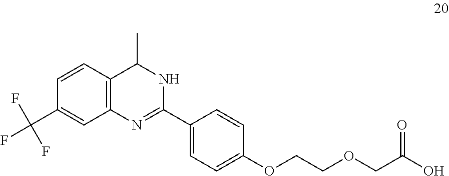 [SMILES: CC1NC(c2ccc(OCCOCC(=O)O)cc2)=Nc2cc(C(F)(F)F)ccc21]

Example 20 was prepared in analogy to the procedure described for the preparation of example 19 by using methyl 2-[2-(p-tolylsulfonyloxy)ethoxy]acetate instead of methyl 3-[2-(p-tolylsulfonyloxy)ethoxy]cyclobutanecarboxylate in step 9. ¹H NMR (400 MHz, MeOH-d4): δ ppm 12.17 (br s, 1H), 10.70-10.85 (m, 1H), 7.87-8.01 (m, 2H), 7.68-7.74 (m, 1H), 7.58-7.64 (m, 2H), 7.23-7.35 (m, 2H), 5.07-5.31 (m, 1H), 4.21-4.36 (m, 2H), 4.03-4.20 (m, 2H), 3.80-3.96 (m, 2H), 1.62 (d, J=6.7 Hz, 3H). MS obsd. (ESI⁺) [(M+H)⁺]:409.1.

### Example 21: cis-3-[2-[4-(8-chloro-4-methyl-3,4-dihydroquinazolin-2-yl)phenoxy]ethoxy]cyclobutanecarboxylic Acid

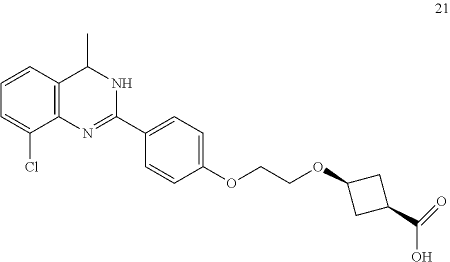 [SMILES: CC1NC(c2ccc(OCCO[C@H]3C[C@@H](C(=O)O)C3)cc2)=Nc2c(Cl)cccc21]

Step 1: Preparation of cis-tert-butyl 3-(2-benzyloxyethoxy)cyclobutanecarboxylate

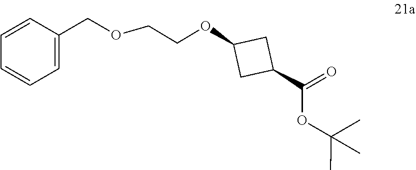 [SMILES: CC(C)(C)OC(=O)[C@H]1C[C@@H](OCCOCc2ccccc2)C1]

To a solution of trifluoromethanesulfonic anhydride (27.8 g, 98.56 mmol) and 2,6-lutidine (11.48 mL, 98.56 mmol) in DCM (100 mL) cooled at −30° C. was added 2-(benzyloxy)ethanol (10.0 g, 65.71 mmol) and the reaction mixture was stirred at −30° C. for 1 hour. The reaction mixture was washed with brine (30 mL) twice and the organic layer was concentrated in vacuo to give the crude 2-(benzyloxy)ethyl trifluoromethanesulfonate (18.7 g, 65.7 mmol) as yellow oil.

To a solution of cis-tert-butyl 3-hydroxycyclobutanecarboxylate (CAS #: 939768-64-6, Cat.#: B253665, from BePharm Ltd., 11.3g, 65.71 mmol) in THF (150 mL) cooled at 0° C. was added NaH (3942.44 mg, 98.56 mmol) and the mixture was stirred at room temperature for 1 hour. To the resulting solution was added freshly prepared 2-(benzyloxy)ethyl trifluoromethanesulfonate (18.7 g, 65.7 mmol) and the mixture was stirred at room temperature for 2 hours. The reaction was then quenched with ice water (100 mL) and the resulting mixture was extracted with EtOAc (200 mL) twice. The combined organic layer was dried over Na₂SO₄ and concentrated in vacuo. The residue was purified by column chromatography on silica gel (elution with PE:EtOAc 100:1 to 2:1) to give cis-tert-butyl 3-(2-benzyloxyethoxy)cyclobutanecarboxylate (10.0 g, 49.67% yield) as a yellow oil. ¹H NMR (CDCl₃, 400 MHz): δ ppm 1.44 (s, 9H), 2.17 (m, 2H), 2.54-2.42 (m, 3H), 3.55-3.50 (m, 2H), 3.62-3.57 (m, 2H), 3.99-3.83 (m, 1H), 4.57 (s, 2H), 7.30-7.27 (m, 1H), 7.34 (d, J=4.3 Hz, 4H). MS obsd. (ESI⁺) [(M+Na)⁺]:329.1.

Step 2: Preparation of cis-tert-butyl 3-[2-(p-tolylsulfonyloxy)ethoxy]cyclobutanecarboxylate

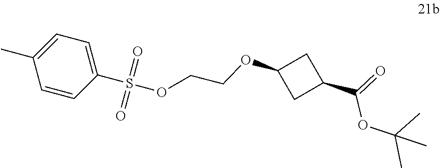 [SMILES: Cc1ccc(S(=O)(=O)OCCO[C@H]2C[C@@H](C(=O)OC(C)(C)C)C2)cc1]

Compound 21b was prepared in analogy to the procedure described for the preparation of compound 19d by using cis-tert-butyl 3-(2-benzyloxyethoxy)cyclobutanecarboxylate as the starting material instead of methyl 3-(2-benzyloxyethoxy)cyclobutanecarboxylate in Step 4. MS obsd. (ESI⁺) [(M+H)⁺]:371.2.

Step 3: Preparation of cis-3-[2-[4-(8-chloro-4-methyl-3,4-dihydroquinazolin-2-yl)phenoxy]ethoxy]cyclobutanecarboxylic Acid

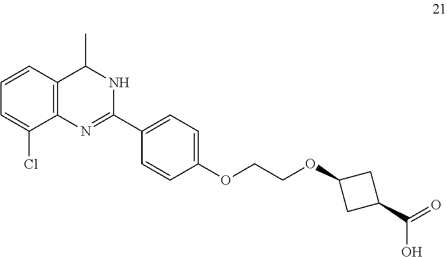 [SMILES: CC1NC(c2ccc(OCCO[C@H]3C[C@@H](C(=O)O)C3)cc2)=Nc2c(Cl)cccc21]

Example 21 was prepared in analogy to the procedure described for the preparation of example 19 by using 1-(2-amino-3-chloro-phenyl)ethanone instead of 1-[2-amino-4-(trifluoromethyl)phenyl]ethanone in step 5 and using cis-tert-butyl 3-[2-(p-tolylsulfonyloxy)ethoxy]cyclobutanecarboxylate instead of methyl 3-[2-(p-tolylsulfonyloxy)ethoxy]cyclobutanecarboxylate in step 9. ¹H NMR (400 MHz, MeOH-d4): δ ppm 7.69-7.84 (m, 2H), 7.41 (dd, J=7.8, 1.5 Hz, 1H), 7.19-7.31 (m, 2H), 7.09-7.17 (m, 2H), 4.97-5.11 (m, 1H), 4.11-4.20 (m, 2H), 3.86-3.98 (m, 1H), 3.63-3.73 (m, 2H), 2.52-2.66 (m, 1H), 2.37-2.51 (m, 2H), 1.93-2.11 (m, 2H), 1.58 ppm (d, J=6.7 Hz, 3H). MS obsd. (ESI⁺) [(M+H)⁺]:414.9.

### Example 22: 3-[2-[4-(8-chloro-4-methyl-3,4-dihydroquinazolin-2-yl)-3-methyl-phenoxy]ethoxy]cyclobutanecarboxylic Acid

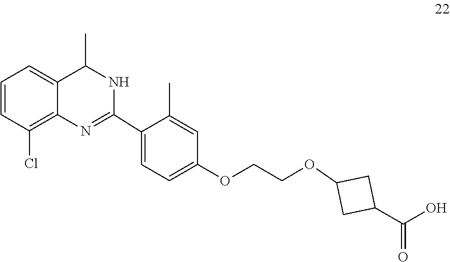 [SMILES: Cc1cc(OCCOC2CC(C(=O)O)C2)ccc1C1=Nc2c(Cl)cccc2C(C)N1]

Step 1: Preparation of 4-(8-chloro-4-methyl-3,4-dihydroquinazolin-2-yl)-3-methyl-phenol

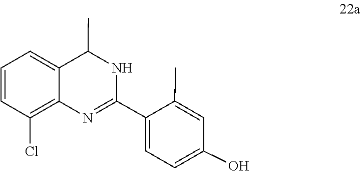 [SMILES: Cc1cc(O)ccc1C1=Nc2c(Cl)cccc2C(C)N1]

Compound 22a was prepared in analogy to the procedure described for the preparation of compound 19g by using 1-(2-amino-3-chloro-phenyl)ethanone and 4-methoxy-2-methyl-benzaldehyde as the starting material instead of 1-[2-amino-4-(trifluoromethyl)phenyl]ethanone and 4-methoxybenzaldehyde in Step 5. MS obsd. (ESI⁺) [(M+H)⁺]:287.1.

Step 2: Preparation of 3-[2-[4-(8-chloro-4-methyl-3,4-dihydroquinazolin-2-yl)-3-methyl-phenoxy]ethoxy]cyclobutanecarboxylic Acid

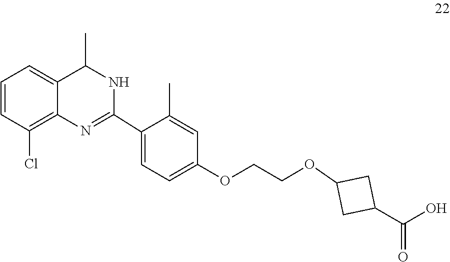 [SMILES: Cc1cc(OCCOC2CC(C(=O)O)C2)ccc1C1=Nc2c(Cl)cccc2C(C)N1]

Example 22 was prepared in analogy to the procedure described for the preparation of example 19 by using 4-(8-chloro-4-methyl-3,4-dihydroquinazolin-2-yl)-3-methyl-phenol instead of 4-[4-methyl-7-(trifluoromethyl)-3,4-dihydroquinazolin-2-yl]phenol in step 8. ¹H NMR (400 MHz, DMSO-d6): δ ppm 7.81-7.92 (m, 1H), 7.31-7.40 (m, 1H), 7.23 (dd, J=7.8, 1.5 Hz, 1H), 6.92-7.08 (m, 2H), 6.82-6.89 (m, 2H), 4.68-4.80 (m, 1H), 4.05-4.21 (m, 2H), 3.85-4.00 (m, 1H), 3.53-3.71 (m, 2H), 2.88-2.96 (m, 1H), 2.45-2.48 (s, 3H), 2.33-2.43 (m, 2H), 2.09-2.21 (m, 1H), 1.90-2.03 (m, 1H), 1.22-1.45 (m, 3H). MS obsd. (ESI⁺) [(M+H)⁺]:429.5.

### Example 23: 2-[2-[4-(8-chloro-4-methyl-3,4-dihydroquinazolin-2-yl)phenoxy]ethoxy]acetic Acid

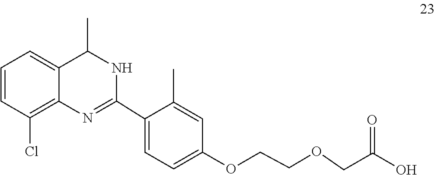 [SMILES: Cc1cc(OCCOCC(=O)O)ccc1C1=Nc2c(Cl)cccc2C(C)N1]

Example 23 was prepared in analogy to the procedure described for the preparation of example 19 by using 4-(8-chloro-4-methyl-3,4-dihydroquinazolin-2-yl)-3-methyl-phenol instead of 4-[4-methyl-7-(trifluoromethyl)-3,4-dihydroquinazolin-2-yl]phenol in step 8 and by using methyl 2-[2-(p-tolylsulfonyloxy)ethoxy]acetate instead of methyl 3-[2-(p-tolylsulfonyloxy)ethoxy]cyclobutanecarboxylate in step 9. ¹H NMR (400 MHz, MeOH-d4): δ ppm 7.74 (d, J=8.9 Hz, 2H), 7.34-7.41 (m, 1H), 7.08-7.28 (m, 4H), 4.96-5.06 (m, 1H), 4.18-4.25 (m, 2H), 3.89-3.96 (m, 2H), 3.77-3.86 (m, 2H), 1.55 (d, J=6.7 Hz, 3H). MS obsd. (ESI⁺) [(M+H)⁺]:375.1.

### Example 24: 2-(3-bromophenyl)-3,4-dimethyl-7-(trifluoromethyl)-4H-quinazoline

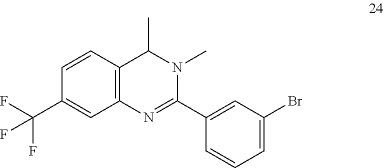 [SMILES: CC1c2ccc(C(F)(F)F)cc2N=C(c2cccc(Br)c2)N1C]

Step 1: Preparation of N-[2-acetyl-5-(trifluoromethyl)phenyl]-3-bromo-benzamide

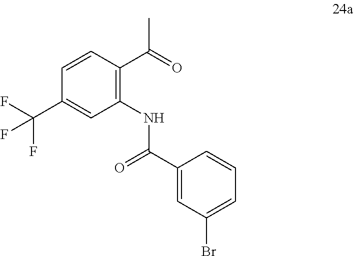 [SMILES: CC(=O)c1ccc(C(F)(F)F)cc1NC(=O)c1cccc(Br)c1]

To a solution of 1-(2-amino-4-(trifluoromethyl)phenyl)ethanone (1.1 g, 5.41 mmol) in DCM (12 mL) were added N-methylmorpholine (1.1 g, 1.19 mL, 10.8 mmol) and 3-bromobenzoyl chloride (1.54 g, 925 μl, 7.04 mmol) at room temperature and the mixture was then stirred at room temperature over night. After the reaction was completed, the mixture was diluted with DCM (20 mL) and washed with water (10 mL). The organic layer was concentrated in vacuo and the residue was purified by column chromatography on silica gel (elution with PE: EtOAc=5:1) to give N-[2-acetyl-5-(trifluoromethyl)phenyl]-3-bromo-benzamide (1.9 g, 90.9% yield) as a light grey solid. ¹H NMR (400 MHz, CDC13): δ ppm 12.75 (br s, 1H), 9.26-9.37 (m, 1H), 8.18-8.29 (m, 1H), 8.06-8.18 (m, 1H), 7.94-8.04 (m, 1H), 7.67-7.80 (m, 1H), 7.35-7.52 (m, 2H), 2.73-2.89 (m, 3H). (ESI⁺) [(M+H)⁺]:387.1.

Step 2: Preparation of 2-(3-bromophenyl)-3,4-dimethyl-7-(trifluoromethyl)-4H-quinazoline

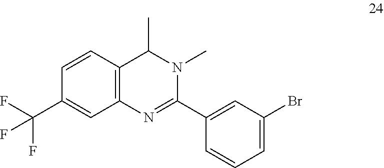 [SMILES: CC1c2ccc(C(F)(F)F)cc2N=C(c2cccc(Br)c2)N1C]

To a solution of N-[2-acetyl-5-(trifluoromethyl)phenyl]-3-bromo-benzamide (100 mg, 259 μmol) in EtOH (4 mL) was added methanamine (1.29 mL, 2.59 mmol) and the mixture was then stirred at 90° C. for 1 hour. LC-MS showed complete conversion of N-(2-acetyl-5-(trifluoromethyl)phenyl)-3-bromobenzamide. To the resulting mixture was added NaBH₄ (98 mg, 2.59 mmol) and the mixture was stirred at 90° C. overnight. The mixture was quenched with AcOH (0.2 mL) and diluted with water (5 mL). The mixture was then extracted with EtOAc (20 mL) three times. The combined organic layer was washed with brine, dried over anhydrous Na₂SO₄ and concentrated in vacuo. The residue was purified by preparative HPLC to give the 2-(3-bromophenyl)-3,4-dimethyl-7-(trifluoromethyl)-4H-quinazoline (29 mg, 28.9% yield) as a colorless semi-solid. ¹H NMR (400 MHz, MeOH-d4): δ ppm 8.42 (s, 1H), 7.78-7.85 (m, 2H), 7.60-7.66 (m, 1H), 7.52-7.56 (m, 1H), 7.46-7.52 (m, 1H), 7.32-7.39 (m, 2H), 4.92-5.01 (m, 1H), 3.14-3.20 (m, 3H), 1.59 (d, J=6.5 Hz, 3H). (ESI⁺) [(M+H)⁺]:383.5.

### Example 25: 2-[2-(3-bromophenyl)-4-methyl-7-(trifluoromethyl)-4H-quinazolin-3-yl]ethanol

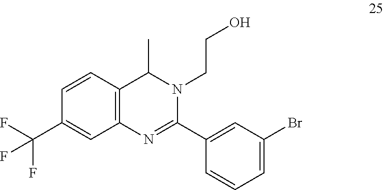 [SMILES: CC1c2ccc(C(F)(F)F)cc2N=C(c2cccc(Br)c2)N1CCO]

Example 25 was prepared in analogy to the procedure described for the preparation of example 24 by using 2-aminoethanol instead of methanamine in step 2. ¹H NMR (400 MHz, MeOH-d4): δ ppm 7.75-7.78 (m, 1H), 7.69-7.73 (m, 1H), 7.55-7.60 (m, 1H), 7.43-7.49 (m, 1H), 7.39-7.43 (m, 1H), 7.34-7.37 (m, 1H), 7.25-7.29 (m, 1H), 4.90-4.95 (m, 1H), 3.59-3.67 (m, 2H), 3.50-3.59 (m, 2H), 3.39-3.46 (m, 1H), 1.46 (d, J=6.5 Hz, 3H). MS obsd. (ESI⁺) [(M+H)⁺]:413.2.

### Example 26: 2-[2-[4-(8-chloro-3,4-dimethyl-4H-quinazolin-2-yl)phenoxy]ethoxy]acetic acid

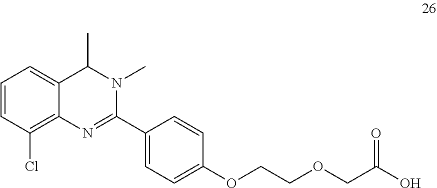 [SMILES: CC1c2cccc(Cl)c2N=C(c2ccc(OCCOCC(=O)O)cc2)N1C]

Step 1: Preparation of 8-chloro-2-(4-methoxyphenyl)-3,4-dimethyl-4H-quinazoline

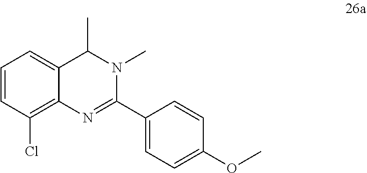 [SMILES: COc1ccc(C2=Nc3c(Cl)cccc3C(C)N2C)cc1]

Compound 26a was prepared in analogy to the procedure described for the preparation of example 24 by using 1-(2-amino-3-chloro-phenyl)ethanone and 4-methoxybenzoyl chloride instead of 1-(2-amino-4-(trifluoromethyl)phenyl)ethanone and 3-bromobenzoyl chloride in step 1. MS obsd. (ESI⁺) [(M+H)⁺]:301.2.

Step 2: Preparation of 4-(8-chloro-3,4-dimethyl-4H-quinazolin-2-yl)phenol

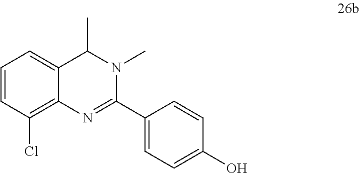 [SMILES: CC1c2cccc(Cl)c2N=C(c2ccc(O)cc2)N1C]

A solution of 8-chloro-2-(4-methoxyphenyl)-3,4-dimethyl-4H-quinazoline (200 mg, 0.67 mmol) in concentrated HBr acid (5 mL) was stirred at 140° C. for 5 hours. After the reaction was completed, the mixture was then concentrated in vacuo to give the crude of 4-(8-chloro-3,4-dimethyl-4H-quinazolin-2-yl)phenol (190 mg, 100% yield), which was used in the next step directly without further purification. MS obsd. (ESI⁺) [(M+H)⁺]:287.6.

Step 3: Preparation of methyl 2-[2-[4-(8-chloro-3,4-dimethyl-4H-quinazolin-2-yl)phenoxy]ethoxy]acetate

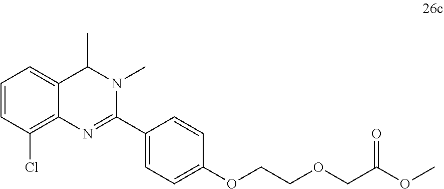 [SMILES: COC(=O)COCCOc1ccc(C2=Nc3c(Cl)cccc3C(C)N2C)cc1]

To a mixture of 4-(8-chloro-3,4-dimethyl-4H-quinazolin-2-yl)phenol (60 mg, 209 μmol) and methyl 2-(2-(tosyloxy)ethoxy)acetate (72.4 mg, 251 μmol) in DMF (5 mL) was added K₂CO₃ (57.8 mg, 418 μmol) and the mixture was stirred at 80° C. for 2 hours. The mixture was then diluted with water (15 mL) and extracted with EtOAc (20 mL) three times. The combined organic layer was then concentrated in vacuo to give the crude of methyl 2-[2-[4-(8-chloro-3,4-dimethyl-4H-quinazolin-2-yl)phenoxy]ethoxy]acetate (84.3 mg, 100% yield) as a yellow solid, which was used in the next step directly without further purification. MS obsd. (ESI⁺) [(M+H)⁺]:403.1.

Step 4: Preparation of 2-[2-[4-(8-chloro-3,4-dimethyl-4H-quinazolin-2-yl)phenoxy]ethoxy]acetic Acid

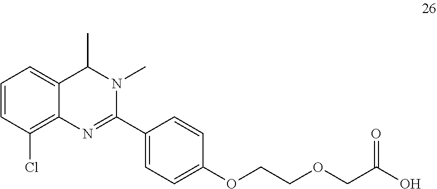 [SMILES: CC1c2cccc(Cl)c2N=C(c2ccc(OCCOCC(=O)O)cc2)N1C]

To a solution of methyl 2-[2-[4-(8-chloro-3,4-dimethyl-4H-quinazolin-2-yl)phenoxy]ethoxy]acetate (50 mg, 124 μmol) in a mixed solvent of THF (4 mL) and water (1 mL) was added LiOH (14.9 mg, 621 μmol). The mixture was stirred at room temperature overnight. After the reaction was completed, the reaction was quenched by addition of AcOH (60 mg) and concentrated in vacuo. The residue was then purified by preparative HPLC to give 2-[4-[4-(8-chloro-3,4-dimethyl-4H-quinazolin-2-yl)phenoxy]ethoxy]acetic acid (15 mg, 31.1% yield) as a white solid. ¹H NMR (400 MHz, MeOH-d4): δ ppm 11.76-12.00 (m, 1H), 7.72-7.81 (m, 2H), 7.51-7.60 (m, 1H), 7.37 (s, 1H), 7.28-7.33 (m, 1H), 7.21-7.29 (m, 2H), 6.46-6.66 (m, 1H), 5.03-5.22 (m, 1H), 4.23-4.32 (m, 2H), 3.69-3.92 (m, 2H), 3.31-3.34 (s, 3H), 1.53-1.60 (m, 3H). MS obsd. (ESI⁺) [(M+H)⁺]:389.2.

### Example 27: 2-[4-(8-chloro-3,4-dihydroquinazolin-2-yl)phenoxy]ethanol

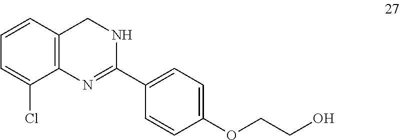 [SMILES: OCCOc1ccc(C2=Nc3c(Cl)cccc3CN2)cc1]

Step 1: Preparation of 4-(8-chloroquinazolin-2-yl)phenol

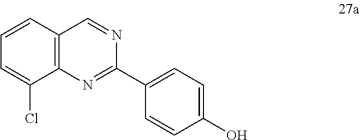 [SMILES: Oc1ccc(-c2ncc3cccc(Cl)c3n2)cc1]

A mixture of 2-amino-3-chlorobenzaldehyde (467 mg, 3 mmol), and 4-hydroxybenzimidamide hydrochloride (518 mg, 3 mmol) in NMP (5 mL) was stirred at 190° C. for 2 hours. After cooling to room temperature, the mixture was poured into water (30 mL) and the resulting suspension was filtred. The filter cake was collected and dried in vacuo to give 4-(8-chloroquinazolin-2-yl)phenol (770 mg, 100% yield) as a white solid, which was used directly in the next step without further purification. MS obsd. (ESI⁺) [(M₊H)⁺]:257.1.

Step 2: Preparation of methyl 2-[4-(8-chloroquinazolin-2-yl)phenoxy]acetate

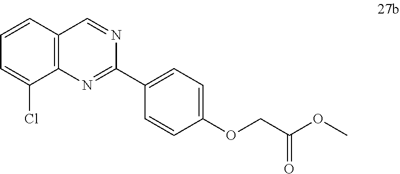 [SMILES: COC(=O)COc1ccc(-c2ncc3cccc(Cl)c3n2)cc1]

To a mixture of 4-(8-chloroquinazolin-2-yl)phenol (770 mg, 3 mmol) and K₂CO₃ (622 mg, 4.5 mmol) in ACN (10 mL) was added methyl 2-bromoacetate (459 mg, 3 mmol) and the mixture was stirred at 80° C. for 3 hours. The mixture was then filtred and the filtrate was concentrated in vacuo, the residue was purifed by column chromatography on silical (elution with PE:EtOAc=100:10˜100:50) to give methyl 2-[4-(8-chloroquinazolin-2-yl)phenoxy]acetate (220 mg, 22.3% yield) as a light yellow solid. MS obsd. (ESI⁺) [(M+H)⁺]:329.1.

Step 3: Preparation of 2-[4-(8-chloro-3,4-dihydroquinazolin-2-yl)phenoxy]ethanol

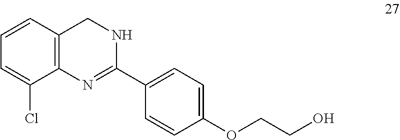 [SMILES: OCCOc1ccc(C2=Nc3c(Cl)cccc3CN2)cc1]

To a solution of methyl 2-[4-(8-chloroquinazolin-2-yl)phenoxy]acetate (200 mg, 608 μmol) in THF (5 mL) was added LiAlH₄ (34.6 mg, 913 μmol) at 0° C. and the mixture was stirred at 0° C. for 2 hours. The reaction was then quenched by addition of 15% NaOH solution 0.5 mL. The resulting mixture was then filtered and the solid was washed with EtOAc (10 mL) twice. The combined filtrate was concentrated in vacuo, the residue was purifed by column chromatography on silical (elution with PE:EtOAc=10:1˜1:10) to give 2-[4-(8-chloro-3,4-dihydroquinazolin-2-yl)phenoxy]ethanol (80 mg, 42.6% yield) as a white solid. ¹H NMR (400 MHz, MeOH-d4): δ ppm 7.95-8.01 (m, 2H), 7.85-7.91 (m, 1H), 7.23 (dd, J=7.3, 2.1 Hz, 1H), 6.99-7.06 (m, 2H), 6.86-6.97 (m, 2H), 4.88-4.94 (m, 1H), 4.55-4.64 (m, 2H), 4.02-4.10 (m, 2H), 3.70-3.77 (m, 2H). MS obsd. (ESI⁺) [(M+H)⁺]:303.1.

### Example 28: 2-[4-(8-chloro-4-methyl-3,4-dihydroquinazolin-2-yl)phenoxy]ethanol

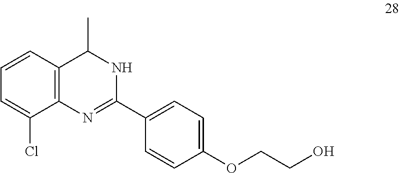 [SMILES: CC1NC(c2ccc(OCCO)cc2)=Nc2c(Cl)cccc21]

Step 1: Preparation of N-(2-acetyl-6-chloro-phenyl)-2,2,2-trichloro-acetamide

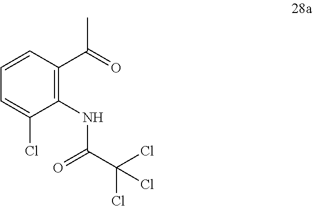 [SMILES: CC(=O)c1cccc(Cl)c1NC(=O)C(Cl)(Cl)Cl]

To a solution of 1-(2-amino-3-chlorophenyl)ethanone (5.5 g, 32.4 mmol) and DMAP (0.7 g, 5.73 mmol) in DCM (50 mL) was added 2,2,2-trichloroacetyl chloride (7.08 g, 38.9 mmol) dropwise at 0° C. After addition, the mixture was stirred at 0° C. for another 2 hours. The mixture was then diluted with EtOAc (100 mL) and the resulting solution washed with water (30 mL) and 0.5 N HCl (30 mL), brine (30 mL) in sequence. The organic layer was separated out, dried over anhydrous Na₂SO₄ and concentrated in vacuo, the residue was then purifed by column chromatography on silical (elution with PE:EtOAc=10:1˜2:1) to give N-(2-acetyl-6-chloro-phenyl)-2,2,2-trichloro-acetamide (9.8 g, 98.2% yield) as a light yellow solid.

Step 2: Preparation of 8-chloro-4-methyl-1H-quinazolin-2-one

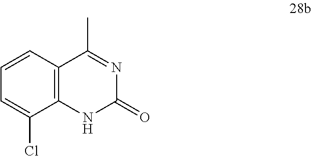

To a solution of N-(2-acetyl-6-chloro-phenyl)-2,2,2-trichloro-acetamide (9.8 g, 31.2 mmol) in THF (20 mL) and was added to ammonium hydroxide (13.5 g, 385 mmol). The mixture was stirred at 40° C. overnight. The mixture was then concentrated in vacuo to give the crude of 8-chloro-4-methyl-1H-quinazolin-2-one (6.1 g, 100% yield) as a white solid, which was used in the next step directly without further purification. MS obsd. (ESI⁺) [(M+H)⁺]:195.4.

Step 3: Preparation of 2,8-dichloro-4-methyl-quinazoline

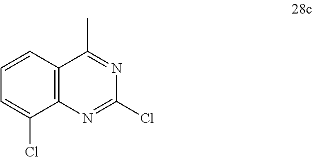

To a suspension solution of 8-chloro-4-methyl-1H-quinazolin-2-one (3.5 g, 18 mmol) in POC13 (19.7 g, 12 mL, 129 mmol) was added N,N-diethylaniline (2.68 g, 18 mmol) and the mixture was stirred at 80° C. for 3 hours. After cooling to room temperature, the mixture was quenched with ice-water carefully. The resulting mixture was then extracted with DCM (30 mL) three times. The combined organic layer was concentrated in vacuo, the residue was purifed by column chromatography on silical (elution with PE:EtOAc=10:1˜2:1) to give 2,8-dichloro-4-methyl-quinazoline (2.4 g, 62.6% yield) as a light yellow solid. MS obsd. (ESI⁺) [(M+H)⁺]:211.1.

Step 4: Preparation of 2-[4-(8-chloro-4-methyl-quinazolin-2-yl)phenoxy]ethanol

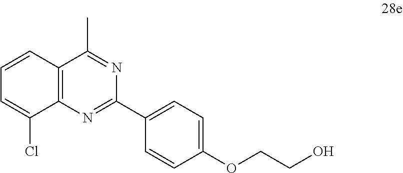 [SMILES: Cc1nc(-c2ccc(OCCO)cc2)nc2c(Cl)cccc12]

A mixture of 2,8-dichloro-4-methyl-quinazoline (800 mg, 3.75 mmol), 2-(4-(4,4,5,5-tetramethyl-1,3,2-dioxaborolan-2-yl)phenoxy)ethanol (992 mg, 3.75 mmol), Pd(Ph₃P)₄ (130 mg, 113 μmol) and Cs₂CO₃ (1.84 g, 5.63 mmol) in a mixed solvent of toluene (7 mL), MeOH (1 mL) and water (1 mL) was charged with N2 and stirred at 100° C. under microwave condition for 3 hours. The reaction was then diluted with water (10 mL) and extracted with EtOAc (20 mL) three times. The combined organic layer was concentrated in vacuo and the residue was purifed by column chromatography on silical (elution with PE:EtOAc=10:1˜2:1) to give 2-[4-(8-chloro-4-methyl-quinazolin-2-yl)phenoxy]ethanol (500 mg, 46.5% yield) as a light yellow solid. MS obsd. (ESI⁺) [(M+H)⁺]:315.1.

Step 5: Preparation of 2-[4-(8-chloro-4-methyl-3,4-dihydroquinazolin-2-yl)phenoxy]ethanol

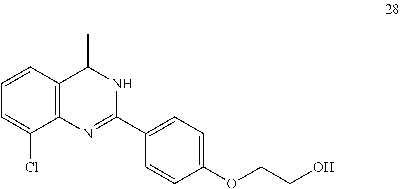 [SMILES: CC1NC(c2ccc(OCCO)cc2)=Nc2c(Cl)cccc21]

To a solution of 2-[4-(8-chloro-4-methyl-quinazolin-2-yl)phenoxy]ethanol (200 mg, 636 μmol) in THF (5 mL) was added LiAlH₄ (34.6 mg, 913 μmol) at 0° C. and the mixture was stirred at 0° C. for 2 hours. The reaction was then quenched by addition of 15% NaOH solution 0.5 mL. The resulting mixture was then filtered and the solid was washed with EtOAc (10 mL) twice. The combined filtrate was concentrated in vacuo, the residue was purifed by column chromatography on silical (elution with PE:EtOAc=10:1˜1:10) to give 2-[4-(8-chloro-4-methyl-3,4-dihydroquinazolin-2-yl)phenoxy]ethanol (105 mg, 52.2% yield) as a white solid. ¹H NMR (400 MHz, DMSO-d6): δ ppm 7.96-8.08 (m, 3H), 7.18-7.30 (m, 1H), 6.98-7.09 (m, 3H), 6.90-6.97 (m, 1H), 4.89-4.93 (m, 1H), 4.70-4.81 (m, 1H), 4.04-4.12 (m, 2H), 3.65-3.80 (m, 2H), 1.33 (d, J=6.4 Hz, 3H). MS obsd. (ESI⁺) [(M+H)⁺]:317.1.

### Example 29: 2-(8-chloro-4-methyl-3,4-dihydroquinazolin-2-yl)-6-methoxy-3,4-dihydroisoquinolin-1-one

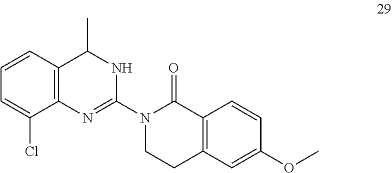 [SMILES: COc1ccc2c(c1)CCN(C1=Nc3c(Cl)cccc3C(C)N1)C2=O]

Step 1: Preparation of 2-(8-chloro-4-methyl-quinazolin-2-yl)-6-methoxy-3,4-dihydroisoquinolin-1-one

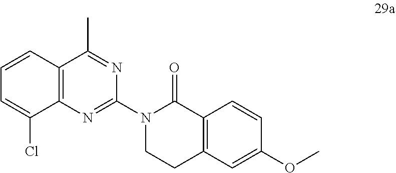 [SMILES: COc1ccc2c(c1)CCN(c1nc(C)c3cccc(Cl)c3n1)C2=O]

A mixture of 2,8-dichloro-4-methylquinazoline (100 mg, 469 μmol), 6-methoxy-3,4-dihydroisoquinolin-1(2H)-one (83.2 mg, 469 μmol), Cs₂CO₃ (459 mg, 1.41 mmol), Pd₂(dba)₃(86 mg, 93.9 μmol), S-Phos (57.7 mg, 141 μmol) in dioxane (3 mL) was stirred at 85° C. under microwave condition for 1.5 hours. After the reaction was completed, the mixture was diluted with water (15 mL) and extracted with EtOAc (20 mL) twice. The combined organic layer was washed with bring (10 mL), dried over Na₂SO₄ and concentrated in vacuo. The residue was purified by column chromatography on silical (elution with PE:EtOAc=10:1˜1:10) to give 2-(8-chloro-4-methyl-quinazolin-2-yl)-6-methoxy-3,4-dihydroisoquinolin-l-one (70 mg, 42.6% yield) as a yellow solid. MS obsd. (ESI⁺) [(M+H)⁺]:354.7.

Step 2: Preparation of 2-(8-chloro-4-methyl-3,4-dihydroquinazolin-2-yl)-6-methoxy-3,4-dihydroisoquinolin-1-one

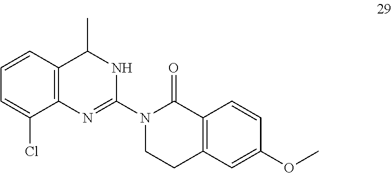 [SMILES: COc1ccc2c(c1)CCN(C1=Nc3c(Cl)cccc3C(C)N1)C2=O]

To a solution of 2-(8-chloro-4-methyl-quinazolin-2-yl)-6-methoxy-3,4-dihydroisoquinolin-1-one (50 mg, 141 μmol) in MeOH was added NaBH₄ (5.35 mg, 141 μmol) and the mixture was then stirred at room temperature for 2 hours. After the reaction was completed, the mixture was purifed by preparative HPLC to give 2-(8-chloro-4-methyl-3,4-dihydroquinazolin-2-yl)-6-methoxy-3,4-dihydroisoquinolin-l-one (5.8 mg, 11.3% yield) as a white solid. ¹H NMR (400 MHz, DMSO-d6): δ ppm 9.49-9.74 (m, 1H), 7.95-8.21 (m, 1H), 6.85-7.06 (m, 4H), 6.55-6.83 (m, 2H), 4.83-4.98 (m, 1H), 4.17-4.72 (s, 3H), 3.75-4.02 (m, 4H), 2.74-3.12 (m, 3H), 1.59-1.83 (m, 2H). MS obsd. (ESI⁺) [(M+H)⁺]:355.4.

### Example 30: 2-(4-bromoimidazol-1-yl)-4-methyl-7-(trifluoromethyl)-3,4-dihydroquinazoline

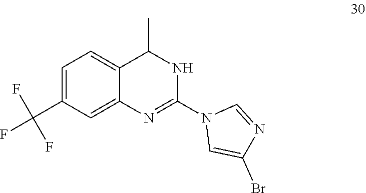 [SMILES: CC1NC(n2cnc(Br)c2)=Nc2cc(C(F)(F)F)ccc21]

Step 1: Preparation of 2-chloro-4-methyl-7-(trifluoromethyl)quinazoline

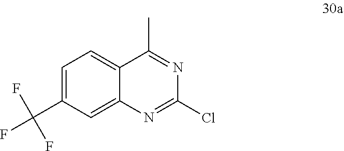 [SMILES: Cc1nc(Cl)nc2cc(C(F)(F)F)ccc12]

Compound 30a was prepared in analogy to the procedure described for the preparation of compound 28c by using 1-(2-amino-4-(trifluoromethyl)phenyl)ethanone instead of 1-(2-amino-3-chlorophenyl)ethanone in step 1. MS obsd. (ESI⁺) [(M+H)⁺]:246.1.

Step 2: Preparation of 2-(4-bromoimidazol-1-yl)-4-methyl-7-(trifluoromethyl)quinazoline

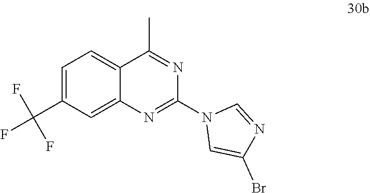 [SMILES: Cc1nc(-n2cnc(Br)c2)nc2cc(C(F)(F)F)ccc12]

To a solution of 2-chloro-4-methyl-7-(trifluoromethyl)quinazoline (200 mg, 811 μmol) in DMF (4 mL) were added 4-bromo-1H-imidazole (179 mg, 1.22 mmol) and K₂CO₃ (112 mg, 811 μmol) at room temperature and the mixture was then stirred at room temperature overnight. After the reaction was completed, the mixture was partitioned between EtOAc (20 mL) and water (20 mL), the organic layer was separated out and the aqueous was extracted with EtOAc (20 mL) twice. The combined organic layer was washed with brine, dried over Na₂SO₄ and concentrated in vacuo. The residue was then purified by column chromatography on silical (elution with PE:EtOAc=10:1˜1:10) to give 2-(4-bromoimidazol-1-yl)-4-methyl-7-(trifluoromethyl)quinazoline (160 mg, 54.7% yield) as a yellow solid. MS obsd. (ESI⁺) [(M+H)⁺]:356.7.

Step 3: Preparation of 2-(4-bromoimidazol-1-yl)-4-methyl-7-(trifluoromethyl)-3,4-dihydroquinazoline

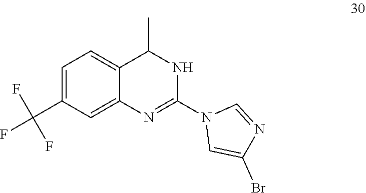 [SMILES: CC1NC(n2cnc(Br)c2)=Nc2cc(C(F)(F)F)ccc21]

To a solution of 2-(4-bromoimidazol-1-yl)-4-methyl-7-(trifluoromethyl)quinazoline (50 mg, 140 μmol) in MeOH (2 mL) was added sodium borohydride (10.6 mg, 280 μmol) at room temperature and the mixture was stirred at room temperature for 40 minutes. After the reaction was completed, the mixture was adjusted to pH-6 by addition of AcOH. The mixture was concentrated in vacuo and the residue was purified by preparative HPLC to give 2-(4-bromoimidazol-1-yl)-4-methyl-7-(trifluoromethyl)-3,4-dihydroquinazoline (22 mg, 39.4% yield) as a white solid. ¹H NMR (400 MHz, DMSO-d6): δ ppm 8.33 (d, J=1.5 Hz, 1H), 7.89 (d, J=1.4 Hz, 1H), 7.34-7.52 (m, 2H), 7.19-7.29 (m, 1H), 5.01 (q, J=6.4 Hz, 1H), 1.43 (d, J=6.5 Hz, 3H). MS obsd. (ESI⁺) [(M+H)⁺]:359.0.

### Example 31: 4-bromo-2-(8-chloro-4-methyl-3,4-dihydroquinazolin-2-yl)thiazole

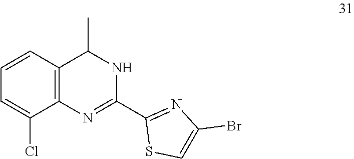 [SMILES: CC1NC(c2nc(Br)cs2)=Nc2c(Cl)cccc21]

Step 1: Preparation of N-(2-acetyl-6-chloro-phenyl)-4-bromo-thiazole-2-carboxamide

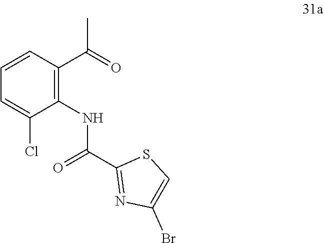 [SMILES: CC(=O)c1cccc(Cl)c1NC(=O)c1nc(Br)cs1]

To a solution of 4-bromothiazole-2-carboxylic acid (1 g, 4.81 mmol) in DCM (20mL) were added oxalyl chloride (1.22 g, 842 μl, 9.61 mmol) and DMF (two drops) at room temperature. The mixture was then stirred at room temperature for 3 hours. The mixture was then concentrated in vacuo to give the crude of 4-bromothiazole-2-carbonyl chloride, which was then re-dissolved in 1,4-dioxane (30 mL) and the solution was added 1-(2-amino-3-chlorophenyl)ethanone (815 mg, 4.81 mmol). The mixture was then stirred at room temperature overnight. After the reaction was completed, the mixture was concentrated in vacuo, the residue was partitioned between EtOAc (30 mL) and saturated NaHCO₃ solution (20 mL). The organic layer was separated out and concentrated in vacuo to give the crude of N-(2-acetyl-6-chloro-phenyl)-4-bromo-thiazole-2-carboxamide (1.5 g, 86.8% yield) as a white solid. MS obsd. (ESI⁺) [(M+H)⁺]:357.9.

Step 2: Preparation of 4-bromo-2-(8-chloro-4-methyl-quinazolin-2-yl)thiazole

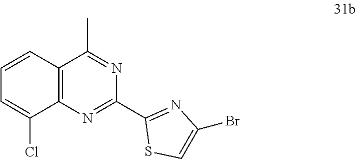 [SMILES: Cc1nc(-c2nc(Br)cs2)nc2c(Cl)cccc12]

The mixture of N-(2-acetyl-6-chloro-phenyl)-4-bromo-thiazole-2-carboxamide (1.5 g, 4.17 mmol) and ammonium acetate (3.22 g, 41.7 mmol) in EtOH (20 mL) was stirred at 80° C. for 7 hours. After the reaction was completed, the mixture was concentrated in vacuo. The residue was then suspended in water (10 mL) and the suspension was then filtred to give 4-bromo-2-(8-chloro-4-methyl-quinazolin-2-yl)thiazole (1.3 g, 91.5% yield) as a white solid. MS obsd. (ESI⁺) [(M+H)⁺]:343.9.

Step 3: Preparation of 4-bromo-2-(8-chloro-4-methyl-3,4-dihydroquinazolin-2-yl)thiazole

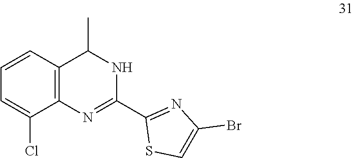 [SMILES: CC1NC(c2nc(Br)cs2)=Nc2c(Cl)cccc21]

To a solution of 4-bromo-2-(8-chloro-4-methyl-quinazolin-2-yl)thiazole (100 mg, 294 μmol) in a mixed solvent of THF (5 mL) and MeOH (2mL) was added NaBH₄ (55.5 mg, 1.47 mmol) at room temperature and the mixture was then stirred at 60° C. for 1.5 hours. After the reaction was completed, the mixture was quenched by addition of NH₄Cl solution (0.3 mL) and the mixture was then concentrated in vacuo. The residue was then triturated with EtOH (5 mL) and the suspension was then filtred. The solid was collected and dried in vacuo to give 4-bromo-2-(8-chloro-4-methyl-3,4-dihydroquinazolin-2-yl)thiazole (48 mg, 46.8% yield) as a white solid. ¹H NMR (400 MHz, DMSO-d6): δ ppm 8.55-8.68 (m, 1H), 8.02-8.11 (m, 1H), 7.25-7.38 (m, 1H), 6.93-7.11 (m, 2H), 4.70-4.89 (m, 1H), 1.35 (d, J=6.4 Hz, 3H). MS obsd. (ESI⁺) [(M+H)⁺]:341.9.

### Example 32: methyl 4-[2-(8-chloro-4-methyl-3,4-dihydroquinazolin-2-yl)thiazol-4-yl]benzoate

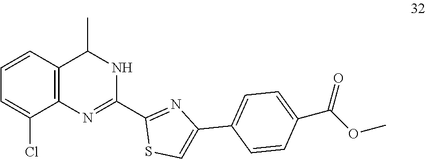 [SMILES: COC(=O)c1ccc(-c2csc(C3=Nc4c(Cl)cccc4C(C)N3)n2)cc1]

Step 1: Preparation of methyl 4-[2-(8-chloro-4-methyl-quinazolin-2-yl)thiazol-4-yl]benzoate

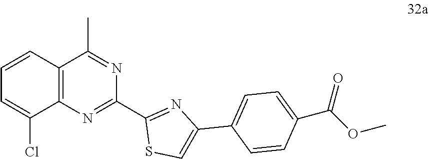 [SMILES: COC(=O)c1ccc(-c2csc(-c3nc(C)c4cccc(Cl)c4n3)n2)cc1]

A mixture of 4-bromo-2-(8-chloro-4-methylquinazolin-2-yl)thiazole (160 mg, 467 μmol), (4-(methoxycarbonyl)phenyl)boronic acid (84 mg, 467 μmol) and Pd(Ph₃P)₄ (27 mg, 23.4 μmol) in the mixed solvent of THF (15 mL) and K₂CO₃ solution (2 mol/L in water, 2.5mL) was stirred at 90° C. for 5hours. After the reaction was completed, THF was removed by concentrated in vacuo, the residue was suspended in EtOAc (8mL). The suspension was then filtred and the solid was collected to give methyl 4-[2-(8-chloro-4-methyl-quinazolin-2-yl)thiazol-4-yl]benzoate (100 mg, 53.8% yield) as a white solid. MS obsd. (ESI⁺) [(M+H)⁺]:400.1.

Step 2: Preparation of methyl 4-[2-(8-chloro-4-methyl-3,4-dihydroquinazolin-2-yl)thiazol-4-yl]benzoate

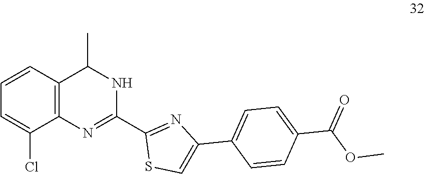 [SMILES: COC(=O)c1ccc(-c2csc(C3=Nc4c(Cl)cccc4C(C)N3)n2)cc1]

To a solution of methyl 4-[2-(8-chloro-4-methyl-quinazolin-2-yl)thiazol-4-yl]benzoate (200 mg, 505 μmol) in a mixed solvent of THF (10 mL) and MeOH (2 mL) was added NaBH₄ (153 mg, 4.04 mmol) at room temperature and the mixture was then stirred at 70° C. for 2hours. After the reaction was completed, the mixture was quenched by addition of NH₄Cl solution (1 mL) and the mixture was then concentrated in vacuo. The residue was then triturated with EtOH (15 mL) and the suspension was then filtred. The solid was collected and dried in vacuo to give methyl 4-[2-(8-chloro-4-methyl-3,4-dihydroquinazolin-2-yl)thiazol-4-yl]benzoate (80 mg, 36.2% yield) as a yellow solid. ¹H NMR (400 MHz, DMSO-d6): δ ppm 8.53-8.59 (m, 2H), 8.24-8.31 (m, 2H), 8.02-8.10 (m, 2H), 7.27-7.34 (m, 1H), 7.00-7.12 (m, 2H), 4.91 (dd, J=6.2, 2.1 Hz, 1H), 3.80-3.93 (m, 3H), 1.41 ppm (d, J=6.4 Hz, 3H). MS obsd. (ESI⁺) [(M+H)⁺]:398.1.

### Example 33: 2-(3-bromophenyl)-4-methyl-7-(trifluoromethyl)-3,4-dihydroquinazoline

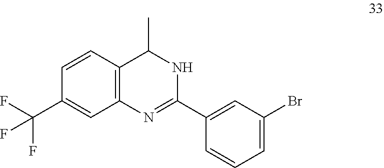 [SMILES: CC1NC(c2cccc(Br)c2)=Nc2cc(C(F)(F)F)ccc21]

Example 33 was prepared in analogy to the procedure described for the preparation of example 31 by using 1-(2-amino-4-(trifluoromethyl)phenyl)ethanone and 3-bromobenzoic acid instead of 1-(2-amino-3-chlorophenyl)ethanone and 4-bromothiazole-2-carboxylic acid in step 1. ¹H NMR (400 MHz, DMSO-d6): δ ppm 8.17 (t, J=1.8 Hz, 1H), 7.95-8.01 (m, 1H), 7.71-7.78 (m, 1H), 7.43-7.51 (m, 1H), 7.17-7.39 (m, 3H), 4.85-4.96 (m, 1H), 1.39 (d, J=6.5 Hz, 3H). MS obsd. (ESI⁺) [(M+H)⁺]:368.9.

### Example 34: 2-(4-bromophenyl)-8-chloro-4,4-dimethyl-3H-quinazoline hydrofluoride

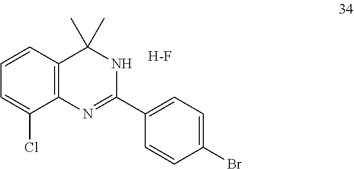

Step 1: Preparation of 2-(2-amino-3-chloro-phenyl)propan-2-ol

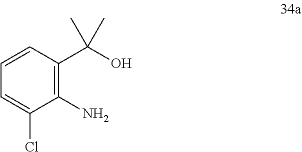

To a solution of methyl 2-amino-3-chlorobenzoate (500 mg, 2.69 mmol) in the THF (10 mL) was added methylmagnesium chloride (3M in THF, 3.59 mL, 10.8 mmol) and the mixture was then stirred at room temperature for 2 hours. After the reaction was completed, the mixture was quenched with saturated NH₄Cl solution (15 mL) and extracted with EtOAc (20 mL) three times. The combined organic layer was washed with brine, dried over Na₂SO₄ and concentrated in vacuo to give the crude of 2-(2-amino-3-chloro-phenyl)propan-2-ol (500 mg, 100% yield) as a colorless oil, which was used in the next step directly without further purification. MS obsd. (ESI⁺) [(M+H)⁺]:186.7.

Step 2: Preparation of 2-(4-bromophenyl)-8-chloro-4,4-dimethyl-3H-quinazoline; hydrofluoride

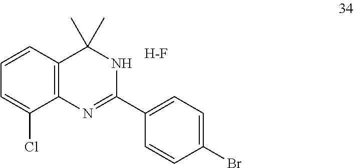

A mixture of 2-(2-amino-3-chloro-phenyl)propan-2-ol (100 mg, 539 μmol) and 4-bromobenzonitrile (98 mg, 539 μmol) in BF₃.Et₂O (0.5 mL) was stirred at 70° C. overnight. The mixture was then concentrated in vacuo and the residue was triturated with MeOH (5 mL). The suspension was then filtred, the solid was collected and dried in vacuo to give 2-(4-bromophenyl)-8-chloro-4,4-dimethyl-3H-quinazoline; hydrofluoride (65 mg, 31.2% yield) as a white solid. ¹H NMR (400 MHz, MeOH-d4): δ ppm 7.88-7.95 (m, 2H), 7.78-7.88 (m, 2H), 7.52-7.58 (m, 1H), 7.45-7.51 (m, 1H), 7.25-7.43 (m, 1H), 1.84 ppm (s, 6H). MS obsd. (ESI⁺) [(M+H)⁺]:349.0.

### Example 35: 2-(4-bromophenyl)-8-chloro-4-cyclopropyl-3,4-dihydroquinazoline

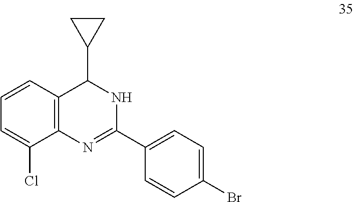 [SMILES: Clc1cccc2c1N=C(c1ccc(Br)cc1)NC2C1CC1]

Step 1: Preparation of (2-amino-3-chloro-phenyl)-cyclopropyl-methanol

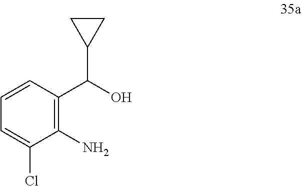 [SMILES: Nc1c(Cl)cccc1C(O)C1CC1]

To a solution of 2-amino-3-chlorobenzaldehyde (500 mg, 3.21 mmol) in the THF (10 mL) was added cyclopropylmagnesium bromide (0.7 mol/L in THF, 18.4 mL, 12.9 mmol), the mixture was then stirred at room temperature for 2 hours. After the reaction was completed, the mixture was quenched with saturated NH₄Cl solution (15 mL) and extracted with EtOAc (20 mL) three times. The combined organic layer was washed with brine, dried over Na₂SO₄ and concentrated in vacuo to give the crude of (2-amino-3-chloro-phenyl)-cyclopropyl-methanol (450 mg, 85% yield) as a colorless oil, which was used in the next step directly without further purification. MS obsd. (ESI⁺) [(M+H)⁺]:197.8.

Step 2: Preparation of 2-(4-bromophenyl)-8-chloro-4-cyclopropyl-3,4-dihydroquinazoline

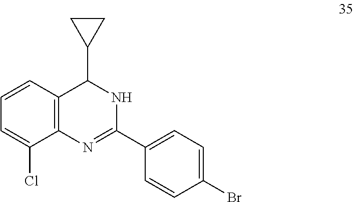 [SMILES: Clc1cccc2c1N=C(c1ccc(Br)cc1)NC2C1CC1]

A mixture of (2-amino-3-chloro-phenyl)-cyclopropyl-methanol (100 mg, 506 μmol) and 4-bromobenzonitrile (92.1 mg, 506 μmol) in BF₃.Et₂O (0.5 mL) was stirred at 70° C. overnight. After the reaction was completed, the mixture was concentrated in vacuo, the residue was purified by preparative HPLC to give 2-(4-bromophenyl)-8-chloro-4-cyclopropyl-3,4-dihydroquinazoline (65 mg, 35.5% yield) as an off-white solid. ¹H NMR (400 MHz, MeOH-d4): δ ppm 7.91 (br d, J=8.4 Hz, 2H), 7.74-7.88 (m, 2H), 7.50-7.60 (m, 1H), 7.31-7.41 (m, 2H), 4.37 (br d, J=8.9 Hz, 1H), 1.30-1.42 (m, 1H), 0.49-0.78 (m, 4H). MS obsd. (ESI⁺) [(M+H)⁺]:361.1.

### Example 36: 2-(4-bromophenyl)-8-chloro-4-isopropyl-3,4-dihydroquinazoline

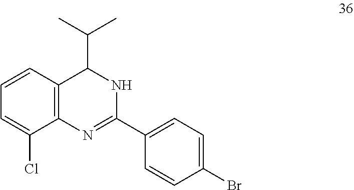 [SMILES: CC(C)C1NC(c2ccc(Br)cc2)=Nc2c(Cl)cccc21]

Example 36 was prepared in analogy to the procedure described for the preparation of example 35 by using isopropylmagnesium bromide instead of cyclopropylmagnesium bromide in step 1. ¹H NMR (400 MHz, MeOH-d4): δ ppm 7.89-8.07 (m, 2H), 7.68-7.86 (m, 2H), 7.24-7.47 (m, 1H), 6.92-7.17 (m, 2H), 4.18-4.71 (m, 1H), 1.68-2.02 (m, 1H), 0.85-0.99 (m, 3H), 0.66-0.84 (m, 3H). MS obsd. (ESI⁺) [(M+H)⁺]:363.1.

### Example 37: 3-[2-[5-bromo-2-(8-chloro-4-methyl-3,4-dihydroquinazolin-2-yl)phenoxy]ethoxy]cyclobutanecarboxylic Acid

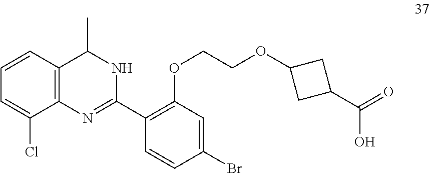 [SMILES: CC1NC(c2ccc(Br)cc2OCCOC2CC(C(=O)O)C2)=Nc2c(Cl)cccc21]

Step 1: Preparation of 1-(2-amino-3-chloro-phenyl)ethanol

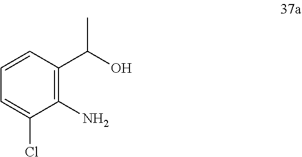

Compound 37a was prepared in analogy to the procedure described for the preparation of compound 35a by using methylmagnesium bromide instead of cyclopropylmagnesium bromide in step 1.

Step 2: Preparation of methyl 3-[2-(5-bromo-2-cyano-phenoxy)ethoxy]cyclobutanecarboxylate

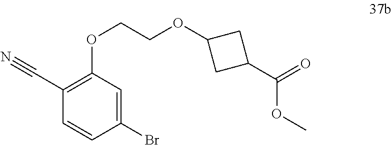 [SMILES: COC(=O)C1CC(OCCOc2cc(Br)ccc2C#N)C1]

To the mixture of 4-bromo-2-hydroxybenzonitrile (1100 mg, 5.56 mmol), K₂CO₃ (1.54 g, 11.1 mmol) in DMF (15 mL) was added methyl 3-(2-(tosyloxy)ethoxy)cyclobutanecarboxylate (1.82 g, 5.56 mmol) and the mixture was then stirred at 60° C. for 5 hours. After the reaction was completed, the mixture was diluted with water (20 mL) and extracted with EtOAc (30 mL) twice. The combined organic layer was washed with brine, dried over Na₂SO₄ and concentrated in vacuo. The residue was then purifed by column chromatography on silical (elution with PE:EtOAc=10:1˜1:10) to give methyl 3-[2-(5-bromo-2-cyano-phenoxy)ethoxy]cyclobutanecarboxylate (1.4 g, 71.2% yield) as a colorless oil.

Step 3: Preparation of methyl 3-[2-[5-bromo-2-(8-chloro-4-methyl-3,4-dihydroquinazolin-2-yl)phenoxy]ethoxy]cyclobutanecarboxylate

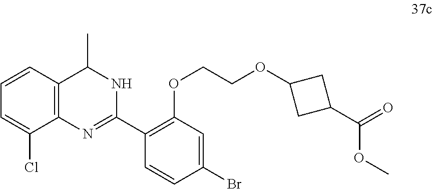 [SMILES: COC(=O)C1CC(OCCOc2cc(Br)ccc2C2=Nc3c(Cl)cccc3C(C)N2)C1]

A mixture of 1-(2-amino-3-chloro-phenyl)ethanol (130 mg, 757 μmol) and methyl 3-[2-(5-bromo-2-cyano-phenoxy)ethoxy]cyclobutanecarboxylate (268 mg, 757 μmol) in (diethyloxonio)trifluoroborate (323 mg, 2.27 mmol) was stirred at 70° C. overnight. After the reaction was completed, the mixture was then quenched with methanol (5 mL) and concentrated in vacuo to give the crude of methyl 3-[2-[5-bromo-2-(8-chloro-4-methyl-3,4-dihydroquinazolin- 2-yl)phenoxy]ethoxy]cyclobutanecarboxylate (385 mg , 100% yield), which was used in the next step directly without further purification. MS obsd. (ESI⁺) [(M+H)⁺]:507.6.

Step 4: Preparation of 3-[2-[5-bromo-2-(8-chloro-4-methyl-3,4-dihydroquinazolin-2-yl)phenoxy]ethoxy]cyclobutanecarboxylic Acid

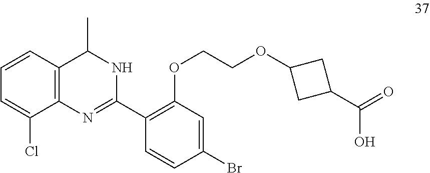 [SMILES: CC1NC(c2ccc(Br)cc2OCCOC2CC(C(=O)O)C2)=Nc2c(Cl)cccc21]

To a solution of methyl 3-[2-[5-bromo-2-(8-chloro-4-methyl-3,4-dihydroquinazolin-2-yl)phenoxy]ethoxy]cyclobutanecarboxylate (100 mg, 197 μmol) in MeOH (3 mL) was added LiOH (18.9 mg, 788 μmol) and the mixture was then stirred at room temperature for 2 hours. After the reaction was completed, the mixture was adjusted to pH-6 by addition of AcOH and concentrated in vacuo. The residue was then purifed by preparative HPLC to give 3-[2-[5-bromo-2-(8-chloro-4-methyl-3,4-dihydroquinazolin-2-yl)phenoxy]ethoxy]cyclobutanecarboxylic acid (85 mg, 83% yield) as a white powder. ¹H NMR (400 MHz, MeOH-d4): δ ppm 7.59-7.69 (m, 1H), 7.45-7.51 (m, 1H), 7.40-7.45 (m, 1H), 7.34-7.39 (m, 1H), 7.16-7.25 (m, 2H), 5.00-5.09 (m, 1H), 4.25-4.35 (m, 2H), 4.15-4.24 (m, 0.6H), 3.88-3.96 (m, 0.4H), 3.67-3.81 (m, 2H), 2.77-2.94 (m, 0.6H), 2.49-2.59 (m, 0.4 H), 2.37-2.47 (m, 2H), 1.96-2.21 (m, 2H), 1.61 (d, J=6.6 Hz, 3H). MS obsd. (ESI⁺) [(M+H)⁺]:493.2.

### Example 38: [2-(4-bromophenyl)-8-chloro-3,4-dihydroquinazolin-4-yl]methanol

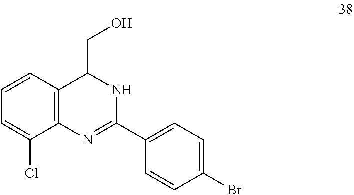 [SMILES: OCC1NC(c2ccc(Br)cc2)=Nc2c(Cl)cccc21]

Step 1: Preparation of ethyl 2-(4-bromophenyl)-8-chloro-quinazoline-4-carboxylate

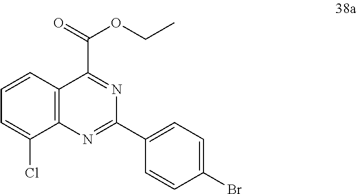 [SMILES: CCOC(=O)c1nc(-c2ccc(Br)cc2)nc2c(Cl)cccc12]

A mixture of compound 1-(4-bromobenzoyl)-7-chloroindoline-2,3-dione (1 g, 2.74 mmol) and ammonium acetate (2.11 g, 27.4 mmol) in ethanol (20 mL) was stirred at 90° C. under microwave condition for 30 minutes. The mixture was then adjusted to pH˜7 by addition of 1N HCl solution. The resulting mixture was extracted twice with EtOAc (60 mL) three times, the combined organic layer was dried over Na₂SO₄, concentrated in vacuo. The residue was purified by column chromatography on silica gel (elution with DCM:MeOH 10:1 to 2:1) to give ethyl 2-(4-bromophenyl)-8-chloro-quinazoline-4-carboxylate (0.4 g, 37.2% yield) as a yellow solid. MS obsd. (ESI⁺) [(M+H)⁺]:391.0.

Step 2: Preparation of [2-(4-bromophenyl)-8-chloro-3,4-dihydroquinazolin-4-yl]methanol

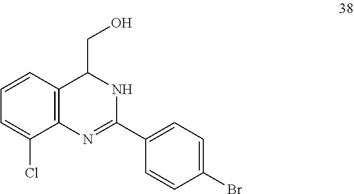 [SMILES: OCC1NC(c2ccc(Br)cc2)=Nc2c(Cl)cccc21]

To a solution of ethyl 2-(4-bromophenyl)-8-chloro-quinazoline-4-carboxylate (50 mg, 128 μmol,) in MeOH (10 mL) was added NaBH₄ (48.3 mg, 1.28 mmol) at room temperature, the mixture was then stirred room temperature for 0.5 hour. After the reaction was completed, the mixture was then adjusted to pH˜7 by addition of AcOH. The mixture was then concentrated in vacuo and the residue was then purified by preparative HPLC to give [2-(4-bromophenyl)-8-chloro-3,4-dihydroquinazolin-4-yl]methanol (40 mg, 89.1% yield) as a white powder. ¹H NMR (400 MHz, MeOH-d4): δ ppm 7.75-8.01 (m, 4H), 7.47-7.66 (m, 1H), 7.29-7.45 (m, 2H), 5.10 (t, J=3.4 Hz, 1H), 3.81-4.07 (m, 2H). MS obsd. (ESI⁺) [(M+H)⁺]:351.2.

### BIOLOGICAL EXAMPLES

### Biological Example 1: Engineered HepDES19 Primary Screen Assay

The assay was employed to screen for novel cccDNA inhibitors. HepDES19 is a cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res.(2006), 72(2), 116-124; Guo, H. et al., J. Virol. (2007), 81(22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per mL. For compound testing, the frozen cells were thawed and seeded into 96-well plates at a density of 6 ×10⁴ cells per well. At 24hours after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (DonJindo, Cat. CK04-20). IC₅₀ values were derived from the dose-response curve using 4 parameter logistic curve fit method.

The compounds of the present invention were tested for their capacity to inhibit extracellular HBeAg level as described herein. The compounds of this invention were found to have IC₅₀ below 50 μM. Particular compounds of formula (I) were found to have IC₅₀ below 5.0 μM Results of HepDES19 primary screen assay are given in Table 1.

TABLE 1
Activity data in HepDES19
primary screen assay

| Example No. | IC₅₀ (μM) |
|---|---|
| 1 | 5.65 |
| 2 | 2.99 |
| 3 | 10.3 |
| 4 | 0.88 |
| 5 | 0.56 |
| 6 | 3.43 |
| 7 | 3.01 |
| 8 | 9.16 |
| 9 | 0.45 |
| 10 | 0.05 |
| 11 | 0.13 |
| 12 | 0.33 |
| 13 | 0.07 |
| 14 | 2.47 |
| 15 | 0.26 |
| 16 | 2.03 |
| 17 | 1.65 |
| 18 | 2.59 |
| 19 | 4.40 |
| 20 | 2.01 |
| 21 | 1.03 |
| 22 | 8.43 |
| 23 | 1.78 |
| 24 | 2.10 |
| 25 | 9.55 |
| 26 | 5.54 |
| 27 | 11.6 |
| 28 | 4.77 |
| 29 | 0.027 |
| 30 | 0.778 |
| 31 | 2.03 |
| 32 | 0.86 |
| 33 | 0.86 |
| 34 | 5.78 |
| 35 | 0.41 |
| 36 | 6.53 |
| 37 | 1.97 |
| 38 | 5.32 |

### Biological Example 2: Cryopreserved Primary Human Hepatocytes (PHH) Assay

This assay is used to confirm the anti-HBV effect of the compounds in HBV PHH infection assay. Cryopreserved PHH (BioreclamationlVT, Lot YJM) was thawed at 37° C. and gently transferred into pre-warmed InVitroGRO HT medium (BioreclamationIVT, Cat. S03317). The mixture was centrifuged at 70 relative centrifugal force (RCF) for 3 minutes at RT, and the supernatant was discarded. Pre-warmed InVitroGRO CP medium (BioreclamationlVT, Cat# S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hours after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), 5 ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hours. The medium was then changed to pre-warmed PHH culture medium containing 4% polyethylene glycol (PEG) MW8000 (Sigma, Cat. P1458-50ML) and 1% DMSO (Sigma, Cat. D2650). 5.8×10⁶ genomic equivalents of HBV were added into the medium.

At 24 hours post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator.

24 hours later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days post-compound treatment, extracellular HBsAg level were measured with Chemiluminescence Immuno Assay (CLIA) kit (Autobio, HBsAg Quantitative CLIA).

HBsAg IC₅₀ values were derived from the dose-response curve using 4 parameter logistic curve fit method. The compounds of formula (I) have HBsAg IC₅₀<20 μM, particularly <1 μM. Results of Cryopreserved PHH assay are given in Table 2.

TABLE 2
HBsAg IC₅₀ data in
Cryopreserved PHH assay

| | HBsAg |
|---|---|
| Example No. | IC₅₀ (μM) |
| 1 | 3.07 |
| 2 | 0.415 |
| 3 | 1.5 |
| 4 | 1.95 |
| 5 | 4.37 |
| 6 | 7.18 |
| 7 | 2.31 |
| 12 | 1.36 |
| 13 | 2.84 |
| 16 | 4.54 |
| 19 | 7.63 |
| 22 | 0.67 |
| 32 | 0.024 |
| 36 | 0.042 |

1. A compound of the formula (I), 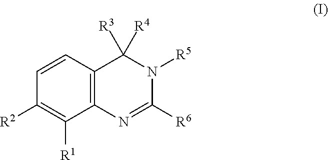
wherein
R¹ is H, halogen, C₁₋₆alkyl or haloC₁₋₆alkyl;
R² is H, halogen, C₁₋₆alkyl or haloC₁₋₆alkyl;
R³ is H, carboxy, C₁₋₆alkyl, haloC₁₋₆alkyl, hydroxyC₁₋₆alkyl, C₁₋₆alkoxycarbonyl, C₃₋₇cycloalkyl, aminocarbonyl, hydroxyC₁₋₆alkylaminocarbonyl, haloC₁₋₆alkylaminocarbonyl or heterocyclylcarbonyl;
R⁴ is H or C₁₋₆alkyl;
R⁵ is H, C₁₋₆alkyl or hydroxyC₁₋₆alkyl;
R⁶ is phenyl or heterocyclyl; wherein phenyl and heterocyclyl are unsubstituted or substituted by one or two or three substituents independently selected from halogen, C₁₋₆alkyl, C₁₋₆alkoxy, hydroxyC₁₋₆alkoxy, C₁₋₆alkoxycarbonylphenyl, carboxyC₁₋₆alkoxyC₁₋₆alkoxy, carboxyC₃₋₇cycloalkylC₁₋₆alkoxy and heterocyclyl;
with the proviso that R¹ and R² are not H simultaneously;
or a pharmaceutically acceptable salt thereof.

2. A compound according to claim 1, wherein
R¹ is H or halogen;
R² is H or haloC₁₋₆alkyl;
R³ is H, carboxy, C₁₋₆alkyl, hydroxyC₁₋₆alkyl, C₁₋₆alkoxycarbonyl, C₃₋₇cycloalkyl, aminocarbonyl, hydroxyC₁₋₆alkylaminocarbonyl, haloC₁₋₆alkylaminocarbonyl or morpholinocarbonyl;
R⁴ is H or C₁₋₆alkyl;
R⁵ is H, C₁₋₆alkyl or hydroxyC₁₋₆alkyl;
R⁶ is phenyl, pyridyl, 1-oxo-3,4-dihydroisoquinolin-2-yl, imidazolyl or thiazolyl; wherein phenyl, pyridyl, 1-oxo-3,4-dihydroisoquinolin-2-yl, imidazolyl and thiazolyl are unsubstituted or substituted by one or two or three substituents independently selected from halogen, C₁₋₆alkyl, C₁₋₆alkoxy, pyrrolidinyl, hydroxyC₁₋₆alkoxy, C₁₋₆alkoxycarbonylphenyl, carboxyC₁₋₆alkoxyC₁₋₆alkoxy and carboxyC₃₋₇cycloalkylC₁₋₆alkoxy;
with the proviso that R¹ and R² are not H simultaneously;
or a pharmaceutically acceptable salt thereof.

3. A compound according to claim 2, wherein
R¹ is H or Cl;
R² is H or CF_{3;}
R³ is H, carboxy, methyl, isopropyl, hydroxymethyl, methoxycarbonyl, cyclopropyl, aminocarbonyl, hydroxyethylaminocarbonyl, chloroethylaminocarbonyl or morpholinocarbonyl;
R⁴ is H or methyl;
R⁵ is H, methyl or hydroxyethyl;
R⁶ is phenyl, pyridyl, 1-oxo-3,4-dihydroisoquinolin-2-yl, imidazolyl or thiazolyl; wherein phenyl, pyridyl, 1-oxo-3,4-dihydroisoquinolin-2-yl, imidazolyl and thiazolyl are unsubstituted or substituted by one or two or three substituents independently selected from Cl, Br, methyl, methoxy, ethoxy, pyrrolidinyl, hydroxyethoxy, methoxycarbonylphenyl, carboxymethoxyethoxy and carboxycyclobutoxyethoxy;
with the proviso that R¹ and R² are not H simultaneously;
or a pharmaceutically acceptable salt thereof.

4. A compound according to claim 1 or 2, or a pharmaceutically acceptable salt thereof, wherein R³ is carboxy, C₁₋₆alkyl or aminocarbonyl.

5. A compound according to claim 4, or a pharmaceutically acceptable salt thereof, wherein R³ is carboxy, methyl, isopropyl or aminocarbonyl.

6. A compound according to any one of claims 1, 2, and 4, or a pharmaceutically acceptable salt thereof, wherein R⁶ is phenyl, imidazolyl or thiazolyl; wherein phenyl, imidazolyl and thiazolyl are unsubstituted or substituted by one or two substituents independently selected from halogen, C₁₋₆alkyl, C₁₋₆alkoxy, C₁₋₆alkoxycarbonylphenyl and carboxyC₃₋₇cycloalkylC₁₋₆alkoxy.

7. A compound according to claim 6, or a pharmaceutically acceptable salt thereof, wherein R⁶ is phenyl, imidazolyl or thiazolyl; wherein phenyl, imidazolyl and thiazolyl are unsubstituted or substituted by one or two substituents independently selected from Br, methyl, methoxy, methoxycarbonylphenyl and carboxycyclobutoxyethoxy.

8. A compound according to any one of claims 1 to 7, or a pharmaceutically acceptable salt thereof, wherein R⁴ is H.

9. A compound according to any one of claims 1 to 8, or a pharmaceutically acceptable salt thereof, wherein R⁵ is H.

10. A compound according to claim 1 or 2 having the formula (II), 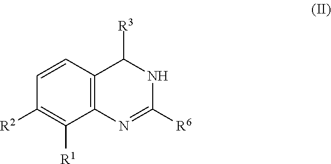
wherein
R¹ is H or halogen;
R² is H or haloC₁₋₆alkyl;
R³ is carboxy, C₁₋₆alkyl or aminocarbonyl;
R⁶ is phenyl, imidazolyl or thiazolyl; wherein phenyl, imidazolyl and thiazolyl are unsubstituted or substituted by one or two substituents independently selected from halogen, C₁₋₆alkyl, C₁₋₆alkoxy, C₁₋₆alkoxycarbonylphenyl and carboxyC₃₋₇cycloalkylC₁₋₆alkoxy;
with the proviso that R¹ and R² are not H simultaneously;
or a pharmaceutically acceptable salt thereof.

11. A compound according to claim 10, wherein
R¹ is H or Cl;
R² is H or CF_{3;}
R³ is carboxy, methyl, isopropyl or aminocarbonyl;
R⁶ is phenyl, imidazolyl or thiazolyl; wherein phenyl, imidazolyl and thiazolyl are unsubstituted or substituted by one or two substituents independently selected from Br, methyl, methoxy, methoxycarbonylphenyl and carboxycyclobutoxyethoxy;
with the proviso that R¹ and R² are not H simultaneously;
or a pharmaceutically acceptable salt thereof.

12. A compound according to any one of claims 1 to 3, selected from
2-(4-bromophenyl)-8-chloro-3,4-dihydroquinazoline-4-carboxylic acid;
8-chloro-2-phenyl-3,4-dihydroquinazoline-4-carboxylic acid;
8-chloro-2-(4-chlorophenyl)-3,4-dihydroquinazoline-4-carboxylic acid;
8-chloro-2-(4-methoxyphenyl)-3,4-dihydroquinazoline-4-carboxylic acid;
2-(4-bromo-3-methyl-phenyl)-8-chloro-3,4-dihydroquinazoline-4-carboxylic acid;
2-(3-bromophenyl)-7-(trifluoromethyl)-3,4-dihydroquinazoline-4-carboxylic acid;
2-(4-bromophenyl)-7-(trifluoromethyl)-3,4-dihydroquinazoline-4-carboxylic acid;
8-chloro-2-(6-methoxy-3-pyridyl)-3,4-dihydroquinazoline-4-carboxylic acid;
8-chloro-2-(6-ethoxy-3-pyridyl)-3,4-dihydroquinazoline-4-carboxylic acid;
8-chloro-2-(6-pyrrolidin-l-yl-3-pyridyl)-3,4-dihydroquinazoline-4-carboxylic acid;
2-(4-bromophenyl)-8-chloro-4-methyl-3H-quinazoline-4-carboxylic acid;
methyl 2-(4-bromophenyl)-8-chloro-3,4-dihydroquinazoline-4-carboxylate;
2-(4-bromophenyl)-8-chloro-3,4-dihydroquinazoline-4-carboxamide;
8-chloro-2-(4-methoxyphenyl)-3,4-dihydroquinazoline-4-carboxamide;
2-(3-bromophenyl)-7-(trifluoromethyl)-3,4-dihydroquinazoline-4-carboxamide;
8-chloro-N-(2-hydroxyethyl)-2-(4-methoxyphenyl)-3,4-dihydroquinazoline-4-carboxamide;
[2-(4-bromophenyl)-7-(trifluoromethyl)-3,4-dihydroquinazolin-4-yl]-morpholino-methanone;
2-(4-bromophenyl)-8-chloro-N-(2-chloroethyl)-3,4-dihydroquinazoline-4-carboxamide;
3-[2-[4-[4-methyl-7-(trifluoromethyl)-3,4-dihydroquinazolin-2-yl]phenoxy]ethoxy]cyclobutanecarboxylic acid;
2-[2-[4-[4-methyl-7-(trifluoromethyl)-3,4-dihydroquinazolin-2-yl]phenoxy]ethoxy]acetic acid;
3-[2-[4-(8-chloro-4-methyl-3,4-dihydroquinazolin-2-yl)phenoxy]ethoxy]cyclobutanecarboxylic acid;
3-[2-[4-(8-chloro-4-methyl-3,4-dihydroquinazolin-2-yl)-3-methyl-phenoxy]ethoxy]cyclobutanecarboxylic acid;
2-[2-[4-(8-chloro-4-methyl-3,4-dihydroquinazolin-2-yl)phenoxy]ethoxy]acetic acid;
2-(3-bromophenyl)-3,4-dimethyl-7-(trifluoromethyl)-4H-quinazoline;
2-[2-(3-bromophenyl)-4-methyl-7-(trifluoromethyl)-4H-quinazolin-3-yl]ethanol;
2-[2-[4-(8-chloro-3,4-dimethyl-4H-quinazolin-2-yl)phenoxy]ethoxy]acetic acid;
2-[4-(8-chloro-3,4-dihydroquinazolin-2-yl)phenoxy]ethanol;
2-[4-(8-chloro-4-methyl-3,4-dihydroquinazolin-2-yl)phenoxy]ethanol;
2-(8-chloro-4-methyl-3,4-dihydroquinazolin-2-yl)-6-methoxy-3,4-dihydroisoquinolin-l-one;
2-(4-bromoimidazol-1-yl)-4-methyl-7-(trifluoromethyl)-3,4-dihydroquinazoline;
4-bromo-2-(8-chloro-4-methyl-3,4-dihydroquinazolin-2-yl)thiazole;
methyl 4-[2-(8-chloro-4-methyl-3,4-dihydroquinazolin-2-yl)thiazol-4-yl]benzoate;
2-(3-bromophenyl)-4-methyl-7-(trifluoromethyl)-3,4-dihydroquinazoline;
2-(4-bromophenyl)-8-chloro-4,4-dimethyl-3H-quinazoline;
2-(4-bromophenyl)-8-chloro-4-cyclopropyl-3,4-dihydroquinazoline;
2-(4-bromophenyl)-8-chloro-4-isopropyl-3,4-dihydroquinazoline;
3-[2-[5-bromo-2-(8-chloro-4-methyl-3,4-dihydroquinazolin-2-yl)phenoxy]ethoxy]cyclobutanecarboxylic acid; and
[2-(4-bromophenyl)-8-chloro-3,4-dihydroquinazolin-4-yl]methanol;
or a pharmaceutically acceptable salt thereof.

13. A compound according to any one of claims 1 to 11, selected from
8-chloro-2-phenyl-3,4-dihydroquinazoline-4-carboxylic acid;
8-chloro-2-(4-methoxyphenyl)-3,4-dihydroquinazoline-4-carboxylic acid;
2-(4-bromophenyl)-8-chloro-3,4-dihydroquinazoline-4-carboxamide;
3-[2-[4-(8-chloro-4-methyl-3,4-dihydroquinazolin-2-yl)-3-methyl-phenoxy]ethoxy]cyclobutanecarboxylic acid;
2-(4-bromoimidazol-1-yl)-4-methyl-7-(trifluoromethyl)-3,4-dihydroquinazoline;
methyl 4-[2-(8-chloro-4-methyl-3,4-dihydroquinazolin-2-yl)thiazol-4-yl]benzoate; and
2-(4-bromophenyl)-8-chloro-4-isopropyl-3,4-dihydroquinazoline;
or a pharmaceutically acceptable salt thereof.

14. A process for the preparation of a compound according to any one of claims 1 to 13 comprising at least one of the following steps:
(a) reduction of a compound of formula (VI), 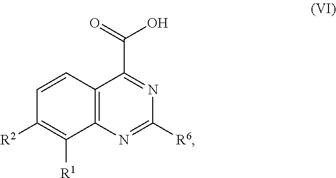
with a reductant;
(b) esterification of a compound of formula (I-1), 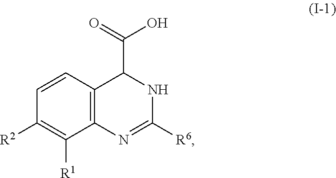
with MeI, in the presence of a base;
(c) reduction of a compound of formula (VII), 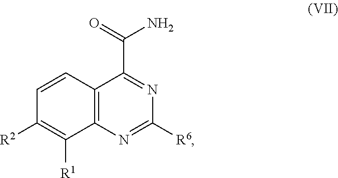
with a reductant;
(d) reduction of a compound of formula (VIII), 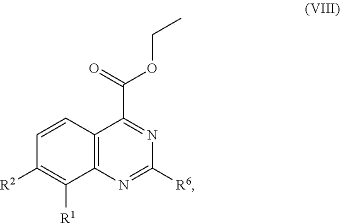
with a reductant;
(e) reduction of a compound of formula (X), 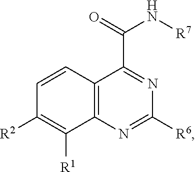
with a reductant;
(f) Deprotection of a compound of formula (XX), 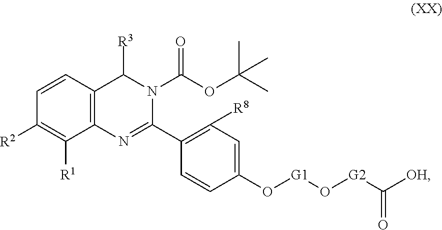
with an acid;
(g) treatment of a compound of formula (XXII), 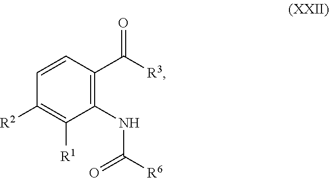
with an amine of formula (A-2), 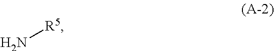
in the presence of a reductant;
(h) reduction of a compound of formula (XXIX), 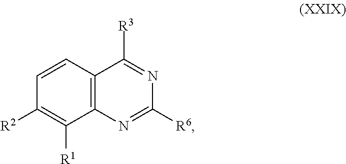
with a reductant;
(i) cyclization of a compound of formula (XXXI), 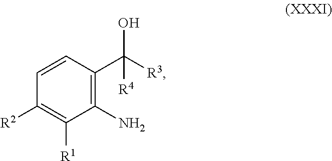
with a compound of formula (XXXII), 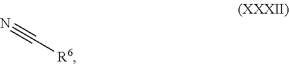
in the presence a Lewis acid;
wherein R⁷ is hydroxyC₁₋₆alkyl or haloC₁₋₆alkyl; R⁸ is hydrogen or C₁₋₆alkyl; R⁹ is C₁₋₆alkyl; G₁ is C₁₋₆alkyl; G₂ is C₁₋₆alkyl or C₃₋₇cycloalkyl; R¹ to R⁶ are defined as in any one of claims 1 to 9.

15. A compound according to any one of claims 1 to 13 for use as therapeutically active substance.

16. A pharmaceutical composition comprising a compound in accordance with any one of claims 1 to 13 and a therapeutically inert carrier.

17. The use of a compound according to any one of claims 1 to 13 for the treatment or prophylaxis of HBV infection.

18. The use of a compound according to any one of claims 1 to 13 for the preparation of a medicament for the treatment or prophylaxis of HBV infection.

19. The use of a compound according to any one of claims 1 to 13 for the inhibition of cccDNA.

20. The use of a compound according to any one of claims 1 to 13 for the inhibition of HBeAg.

21. The use of a compound according to any one of claims 1 to 13 for the inhibition of HBsAg.

22. The use of a compound according to any one of claims 1 to 13 for the inhibition of HBV DNA.

23. A compound according to any one of claims 1 to 13 for use in the treatment or prophylaxis of HBV infection.

24. A compound according to any one of claims 1 to 13, when manufactured according to a process of claim 14.

25. A method for the treatment or prophylaxis of HBV infection, which method comprises administering an effective amount of a compound as defined in any one of claims 1 to 13.
